(19) United States
(12) Patent Application Publication
(10) Pub. No.: US 2024/0293620 A1
(43) Pub. Date: Sep. 5, 2024

(54) ARTICLES, SYSTEMS, AND METHODS FOR THE INJECTION OF VISCOUS FLUIDS

(71) Applicant: Massachusetts Institute of Technology, Cambridge, MA (US)

(72) Inventors: Kripa K. Varanasi, Lexington, MA (US); Vishnu Jayaprakash, Cambridge, MA (US); Simon B. Rufer, Cambridge, MA (US)

(73) Assignee: Massachusetts Institute of Technology, Cambridge, MA (US)

(21) Appl. No.: 18/294,630

(22) PCT Filed: Apr. 11, 2022
(86) PCT No.: PCT/US2022/024218

§ 371 Date: Feb. 2, 2024

Related U.S. Application Data
(60) Provisional application No. 63/229,133, filed on Aug. 4, 2021.

(51) Int. Cl. A61M 5/19; A61M 5/20

(52) U.S. Cl. CPC A61M 5/19; A61M 5/2066; A61M 2206/18

(57) ABSTRACT

Disclosed herein are articles, systems, and methods for the injection of viscous fluids. For example, inventive articles, systems, and methods for injecting viscous fluids, such as concentrated drug formulations, via droplet lubrication are described.

### RELATED APPLICATIONS

This application claims priority under 35 U.S.C. § 119(e) to U.S. Provisional Patent Application No. 63/229,133, filed Aug. 4, 2021, which is hereby incorporated by reference in its entirety.

### TECHNICAL FIELD

Articles, systems, and methods for the injection of viscous fluids are generally described.

### SUMMARY

Disclosed herein are articles, systems, and methods for the injection of viscous fluids. For example, inventive articles, systems, and methods for injecting viscous fluids, such as concentrated drug formulations, via droplet lubrication are described. In some embodiments, injectability of a first fluid (e.g., a concentrated drug formulation) is desired. In certain embodiments, the articles and systems comprise a fluidic pathway comprising an inlet and an outlet and are configured to receive a first fluid and a second fluid, wherein a cross-sectional area of the inlet is larger than a cross-sectional area of the outlet. In certain cases, the second fluid (e.g., a lubricating fluid) lubricates the flow of the first fluid (e.g., a viscous drug) by surrounding the first fluid (e.g., fluid from a first conduit), and the lower viscosity of the second fluid (e.g., fluid from a second conduit) allows the fluid from the first conduit to flow more easily through the system. In some embodiments, the article is configured such that the second fluid axially surrounds the first fluid in the article with an eccentricity parameter of less than 1. In certain embodiments, the article is configured such that the eccentricity parameter of the first and second fluids is lower directly downstream of the outlet than the highest eccentricity parameter at any segment of the article. In some cases, the first fluid does not contact and/or does not substantially contact the interior surface of a needle through which the first fluid is transported. The subject matter of the present disclosure involves, in some cases, interrelated products, alternative solutions to a particular problem, and/or a plurality of different uses of one or more systems and/or articles.

Certain embodiments relate to articles. In some embodiments, the article comprises a fluidic pathway comprising an inlet and an outlet and configured to receive a first fluid and a second fluid; wherein a cross-sectional area of the inlet is larger than a cross-sectional area of the outlet; and wherein the article is configured such that the second fluid axially surrounds the first fluid in the article with an eccentricity parameter of less than 1. In some embodiments, the article is configured such that the eccentricity parameter of the first and second fluids is maintained or lower directly downstream of the outlet than the highest eccentricity parameter at any segment of the article.

In certain embodiments, the article comprises a fluidic pathway comprising an inlet and an outlet and configured to receive a first fluid and a second fluid; wherein a cross-sectional area of the inlet is larger than a cross-sectional area of the outlet; wherein the article is configured such that the second fluid axially surrounds the first fluid in the article; and wherein the article is configured such that the eccentricity parameter of the first and second fluids is maintained or lower directly downstream of the outlet than the highest eccentricity parameter at any segment of the article. In some embodiments, the article is configured such that the second fluid axially surrounds the first fluid in the article with an eccentricity parameter of less than 1.

According to certain embodiments, the eccentricity parameter in the article is less than or equal to 0.9, less than or equal to 0.7, or less than or equal to 0.5.

In accordance with some embodiments, the timescale of convection is less than (e.g., less than or equal to 90%, less than or equal to 70%, or less than or equal to 50% of) the timescale of eccentricity in the article.

In certain embodiments, the article is configured such that the eccentricity parameter of the first and second fluids is greater than or equal to 10% lower, greater than or equal to 50% lower, greater than or equal to 90% lower, or 100% lower directly downstream of the outlet than the highest eccentricity parameter at any segment of the article.

In some embodiments, the difference in the density of the first fluid and the density of the second fluid is less than or equal to 400 kg/m³, less than or equal to 200 kg/m³, less than or equal to 100 kg/m³, or less than or equal to 50 kg/m³.

According to some embodiments, the article comprises one or more constricted regions (optionally with one or more rotational flow generation features and/or obstructions), protrusions on an inner surface, ribs on an inner surface, and/or fins on an inner surface, which, optionally, maintain or lower the eccentricity parameter directly downstream of the outlet compared to the highest eccentricity parameter at any segment of the article.

In accordance with certain embodiments, the article comprises a tapered region, optionally wherein the external angle of the tapered region is less than or equal to 90 degrees (e.g., greater than or equal to 15 degrees and less than or equal to 90 degrees). In certain embodiments, the article has a ratio of L_{HPC}/D_{HO} of less than or equal to 2.

In some embodiments, the article comprises a connector region and has a ratio of L_{CPC}/D_{c} of less than or equal to 2.

According to certain embodiments, the article contains the first fluid and the second fluid, and the length (L) and diameter (D) of at least a portion of the article satisfies the following equation for the first fluid and the second fluid:

$$\frac{L π {D}^{2}}{4 {Q}_{avg} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} < 1$$

where ρₒ is the density of the second fluid, ρᵢ is the density of the first fluid, μₒ is the viscosity of the second fluid, μᵢ is the viscosity of the first fluid, Q_{avg} is the average flowrate of the first fluid and the second fluid, L is the length of the portion of the article, and D is the average diameter of the portion of the article.

In some embodiments, the article contains the first fluid and the second fluid, and the length (L) and diameter (D) of at least a portion of the article satisfies the following equation for the first fluid and the second fluid:

$$\frac{LAi}{{Q}_{i} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$

where ρₒ is the density of the second fluid, ρᵢ is the density of the first fluid, μₒ is the viscosity of the second fluid, μᵢ is the viscosity of the first fluid, Qi is the flowrate of the first fluid through the portion of the article, L is the length of the portion of the article, θ is the angle between the length of the portion of the article and the horizontal plane, g is the gravitational constant, D is the average diameter of the portion of the article, and Aᵢ is determined by the following equations:

$${r}_{i}^{*} = \frac{{r}_{o}}{\sqrt{2 - {μ}_{o} / {μ}_{i}}}$$

$${A}_{i} = {π ( {r}_{i}^{*} )}^{2} ,$$

where rᵢ* is the optimal radius of the first fluid, μₒ is the dynamic viscosity of the second fluid, μᵢ is the dynamic viscosity of the first fluid, rₒ is the radius of the second fluid, and Aᵢ is the cross-sectional area of the first fluid as it flows through the portion of the article.

In certain embodiments, the article contains the first fluid and the second fluid, and the length (L) and diameter (D) of at least a portion of the article satisfies the following equation for the first fluid and the second fluid:

$$\frac{L π {D}^{2}}{4 {Q}_{total} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$

where ρₒ is the density of the second fluid, ρᵢ is the density of the first fluid, μₒ is the viscosity of the second fluid, μᵢ is the viscosity of the first fluid, Qₜₒₜₐₗ is the total flowrate of both fluids through the portion of the article, L is the length of the portion of the article, θ is the angle between the length of the portion of the article and the horizontal plane, g is the gravitational constant, and D is the average diameter of the portion of the article.

Certain embodiments relate to systems. In accordance with some embodiments, the system comprises any article described herein and a needle fluidically connected to the outlet of the article.

In some embodiments, the system comprises any article described herein and a first conduit and a second conduit, wherein the first conduit and second conduit are fluidically connected to the inlet of the article.

In certain embodiments, the system further comprises a needle fluidically connected to the outlet of the article.

According to some embodiments, the first conduit is arranged in a side-by-side configuration with the second conduit.

In accordance with certain embodiments, the system further comprises a chamber comprising a first internal volume and a second internal volume, wherein the first internal volume is fluidically connected to the first conduit and the inlet of the article, and the second internal volume is fluidically connected to the second conduit and the inlet of the article.

In certain embodiments, the second conduit axially surrounds the first conduit.

In some embodiments, the system further comprises: a first plunger associated with the first conduit; and a second plunger associated with the second conduit.

According to certain embodiments, the system further comprises a solid body connecting the first plunger and the second plunger.

In accordance with some embodiments, the system is configured such that when the first plunger and the second plunger are compressed, fluid within the first conduit is transported to the article and fluid within the second conduit is transported to the article such that the fluid from the second conduit at least partially axially surrounds fluid from the first conduit in the article.

In some embodiments, the system is configured such that when the first plunger and the second plunger are compressed, fluid within the first conduit is transported to the needle and fluid within the second conduit is transported to the needle such that the fluid from the second conduit at least partially axially surrounds fluid from the first conduit in the needle.

Certain embodiments relate to methods of delivering one or more fluids using any article and/or system disclosed herein.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is, in accordance with some embodiments, a cross-sectional schematic illustration of an article comprising a fluidic pathway comprising an inlet and an outlet.

FIG. 1B is, in accordance with some embodiments, a cross-sectional schematic illustration of an article comprising a second fluid axially surrounding a first fluid.

FIG. 1C is, in accordance with some embodiments, a cross-sectional schematic illustration of a system comprising an article, a first conduit, and a second conduit, wherein the second conduit axially surrounds the first conduit.

FIG. 1D is, in accordance with some embodiments, a cross-sectional schematic illustration of a system comprising an article, a needle, a chamber, a first conduit, and a second conduit, wherein the first conduit is arranged in a side-by-side configuration with the second conduit.

FIG. 2A is, in accordance with some embodiments, a cross-sectional schematic illustration of a system comprising a needle, an article, a chamber, a first conduit, a second conduit, a first plunger, a second plunger, and a solid body connecting the first and second plungers, wherein the first conduit is arranged in a side-by-side configuration with the second conduit.

FIG. 2B is, in accordance with some embodiments, a cross-sectional schematic illustration of the portion of the article of FIG. 2A shown in the top dotted line rectangle, which comprises a needle, an article, and a chamber.

FIG. 2C is, in accordance with some embodiments, a cross-sectional schematic illustration of the portion of the article of FIG. 2B shown in the dotted line rectangle, which comprises an article. FIG. 2C shows, in accordance with certain embodiments, some dimensions that can be controlled during manufacturing.

FIGS. 2D and 2E show, in accordance with some embodiments, how changing two dimensions from FIG. 2C can, in some cases, affect the ratio of the timescale of convection (T_{c}) and timescale of eccentricity (tₑ) as a function of the density difference between the fluids. A T_{c}/tₑ ratio<1 means that the fluid would pass through the section before eccentricity could fully form. FIG. 2D has an F_{avg} of 4 mm and a D of 1.2 cm, while FIG. 2E has an F_{avg} of 2 mm and a D of 0.25 cm.

FIG. 3A shows, in accordance with some embodiments, a cross-sectional schematic illustration of an article fluidically connected to a needle with definitions of various dimensions of the article and needle shown.

FIG. 3B shows an example of the performance of an article with a D_{HO} of 4 mm, which exhibited eccentric coaxial lubrication (E=1) in the article and needle. In contrast, FIG. 3C shows, in accordance with some embodiments, an example of the performance of an article with a D_{HO} of 2 mm, which exhibited concentric coaxial lubrication (eccentricity mitigation; E=0) in the article and needle. FIG. 4A shows an example, in accordance with some embodiments, of how flow that was partially eccentric in an article became concentric (E=O) in the needle by addition of a constriction region in the article, similar to FIG. 4B, which, in accordance with some embodiments, was concentric throughout the article and needle. FIG. 5A is a cross-sectional view of an example of a needle with an inner fluid and outer fluid in concentric annular flow. FIG. 5B is a cross-sectional view of an example of a needle with an inner fluid and outer fluid in fully eccentric annular flow.

FIG. 5C is a cross-sectional view of an example of a needle with an inner fluid and outer fluid in partially eccentric annular flow.

FIG. 6 is, in accordance with certain embodiments, a schematic illustration of a droplet on a surface within a medium, which can be used to illustrate how the spreading coefficient is determined.

FIG. 7A is, in accordance with certain embodiments, a three dimensional perspective of an interior surface of a needle (and/or article) comprising a texture.

FIG. 7B is, in accordance with certain embodiments, a top view schematic diagram of an interior surface of a needle (and/or article) comprising a texture.

FIG. 8A shows how D is measured when determining the eccentricity parameter, and FIG. 8B shows how D₀ is measured when determining the eccentricity parameter.

### DETAILED DESCRIPTION

Disclosed herein are articles, systems, and methods for the injection of viscous fluids. For example, inventive articles, systems, and methods for injecting viscous fluids, such as concentrated drug formulations, via lubrication are described. In some embodiments, injectability of a first fluid, such as a concentrated drug formulation, is desired. However, the non-linear relationship between formulation concentration and viscosity can greatly limit the ability to inject high concentration drug formulations, which are frequently needed for biologics and/or subcutaneous administration. As drug concentrations increase over 50 mg/mL, the corresponding viscosities frequently range from 20 cP to 1000 cP, making injection through conventional delivery methods (e.g., syringes) extremely challenging. For example, high hydraulic resistance presented by flow through needles at such high concentrations frequently induces large back pressures. In some embodiments, the articles, systems, and/or methods described herein reduce these resistances and enhance the injectability of such high concentration drug formulations, and other high viscosity fluids, by achieving axially lubricated flow with the fluid of interest (e.g., the inner fluid and/or first fluid) and a lubricating fluid (e.g., the outer fluid and/or second fluid).

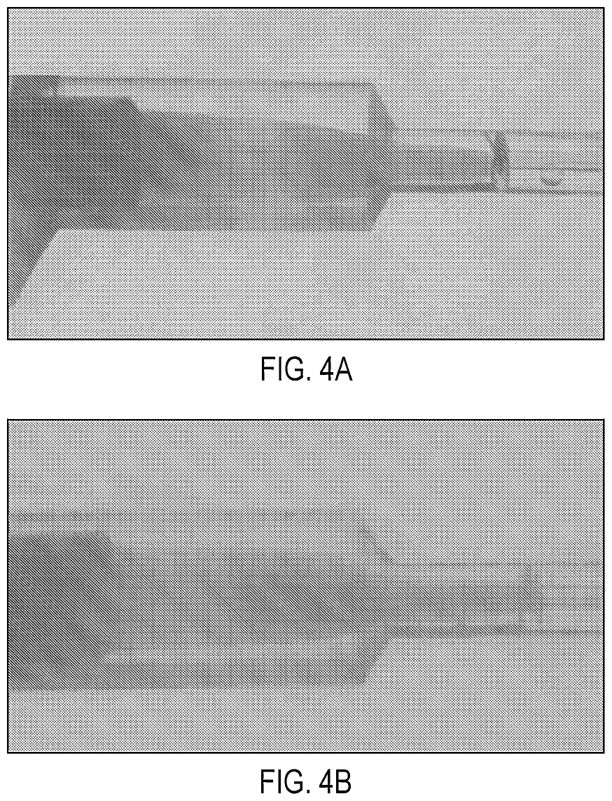

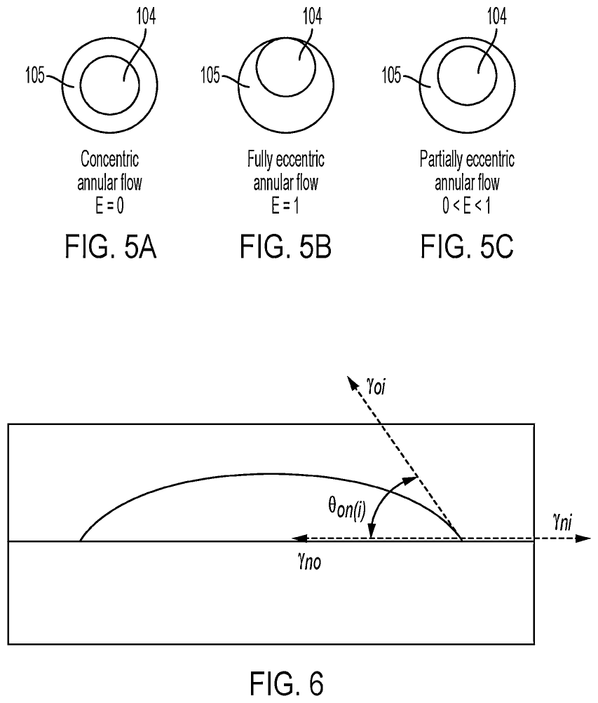

However, axially lubricated flow can be very difficult to achieve in practical systems. For example, eccentricity (e.g., as shown in FIGS. 5B and 5C, compared to a concentric system in FIG. 5A) can arise when the difference in density between the inner fluid and the outer fluid causes a deviation from cylindrical symmetry of the coaxial flow from the centerline of the flow, for example, such that the inner fluid contacts the interior surface of the needle and/or article, reducing the lubrication effect from the outer fluid. However, trying to match the densities of the inner and outer fluids can be extremely impractical in many cases. Avoiding eccentricity can be especially difficult in cases where the outer fluid and inner fluid are miscible. While vertical operation could be used to avoid eccentricity in certain cases, this is also typically impractical, as most subcutaneous injections are not administered vertically. Moreover, vertical operation would only facilitate injection of miscible inner and outer fluids, in certain cases, and would typically not work with immiscible fluids. Certain of the embodiments disclosed herein are capable of achieving axially lubricated flow in practical systems, despite these challenges. For example, in certain embodiments, the articles and systems disclosed herein comprise a fluidic pathway comprising an inlet and an outlet and are configured to receive a first fluid and a second fluid, wherein a cross-sectional area of the inlet is larger than a cross-sectional area of the outlet. In some embodiments, the article is configured such that the second fluid axially surrounds the first fluid in the article with an eccentricity parameter of less than 1. In certain embodiments, the article is configured such that the eccentricity parameter of the first and second fluids is lower directly downstream of the outlet than the highest eccentricity parameter at any segment of the article. Other concepts useful for achieving axially lubricated flow are also disclosed in International Patent Application No. PCT/US2021/015397, filed Jan. 28, 2021, and published as International Patent Application Publication No. WO 2021/154927 on Aug. 5, 2021, which is hereby incorporated by reference in its entirety for all purposes.

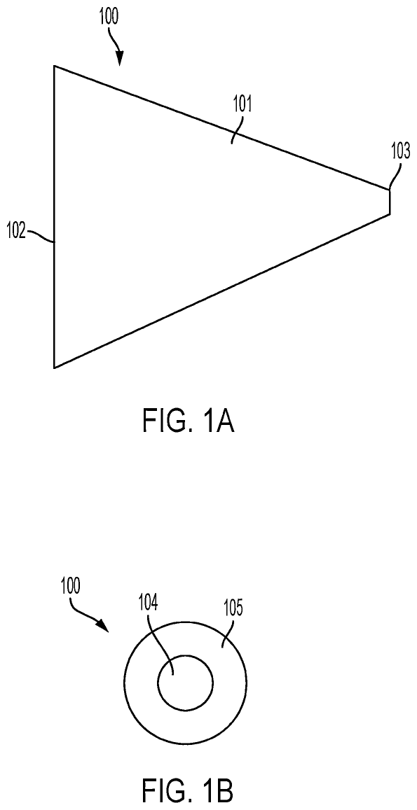

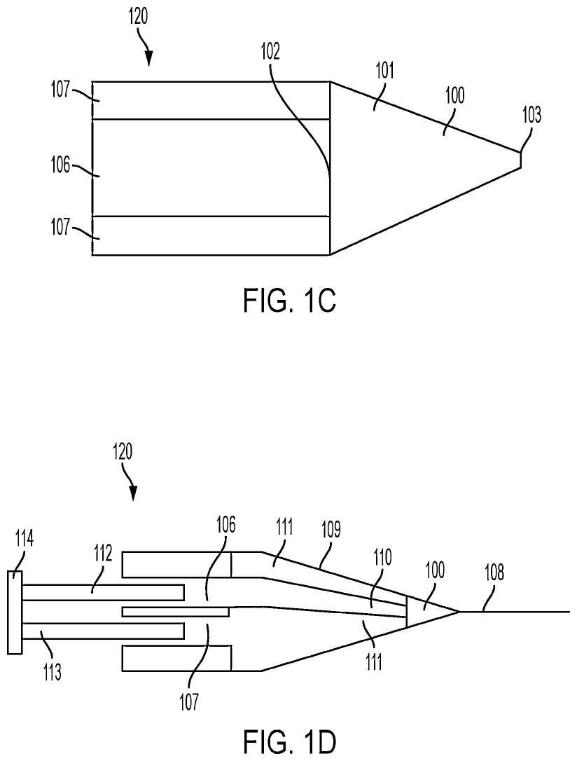

Articles (e.g., for delivery of a fluid) are described herein. One such article is illustrated schematically in FIGS. 1A-1B.

In some embodiments, the article comprises a fluidic pathway. For example, in

FIG. 1A, in certain embodiments, article 100 comprises fluidic pathway 101. According to some embodiments, the fluidic pathway comprises an inlet and/or an outlet. For example, in FIG. 1A, in some embodiments, fluidic pathway 101 comprises inlet 102 and outlet 103. In accordance with some embodiments, a cross-sectional area of the inlet (e.g., a largest cross-sectional area) is larger than a cross-sectional area (e.g., a largest cross-sectional area) of the outlet (e.g., at least 10%, at least 25%, at least 50%, at least 75%, at least 100%, or at least 200% larger).

In accordance with certain embodiments, the fluidic pathway is configured to receive a first fluid (e.g., any first fluid, fluid from a first conduit, and/or inner fluid disclosed herein) and a second fluid (e.g., any second fluid, fluid from a second conduit, and/or outer fluid disclosed herein). For example, in FIG. 1B, in some embodiments, article 100 is configured to receive first fluid 104 and second fluid 105. Examples of fluids include liquids, such as pure liquids and mixtures of liquids, as well as liquids combined with non-liquids, such as liquid/gas mixtures and liquid/solid mixtures, such as suspensions.

In some embodiments, the article is configured such that the second fluid axially surrounds the first fluid in the article. For example, in FIG. 1B, in certain embodiments, article 100 is configured such that second fluid 105 axially surrounds first fluid 104. In certain embodiments, the article is configured such that the second fluid axially surrounds the first fluid in the article with an eccentricity parameter of less than 1 (e.g., less than or equal to 0.9, less than or equal to 0.8, less than or equal to 0.7, less than or equal to 0.6, less than or equal to 0.5, less than or equal to 0.3, less than or equal to 0.2, less than or equal to 0.1, or 0) for at least a portion (e.g., at least 10%, at least 25%, at least 50%, at least 75%, at least 90%, or all) of the article. As shown in FIGS. 5A-5C, an eccentricity parameter of 1 represents fully eccentric annular flow, an eccentricity parameter of 0 represents fully concentric annular flow, and an eccentricity parameter of less than 1 and greater than 0 represents partially eccentric annular flow. A second fluid is said to “axially surround” a first fluid when a continuous pathway can be traced, within the second fluid, that surrounds the longitudinal axis of the first fluid.

The eccentricity parameter (E) may be determined according to the following equation: E=D/D₀ where D is the distance between the geometric center of the inner fluid and the geometric center of the conduit in which the combined flow is flowing (see the line in FIG. 8A), and D₀ is the smallest distance between (1) the geometric center of the inner fluid when the inner fluid is in contact with the wall of the conduit and (2) the geometric center of the conduit (see the line in FIG. 8B).

In accordance with some embodiments, the article is configured such that the eccentricity parameter of the first and second fluids is maintained or lower directly downstream of the outlet than the highest eccentricity parameter at any segment of the article. For example, in some cases, the article is configured such that the eccentricity parameter of the first and second fluids is greater than or equal to 10% lower, greater than or equal to 50% lower, greater than or equal to 90% lower, or 100% lower directly downstream of the outlet than the highest eccentricity parameter at any segment of the article. As an example, if the eccentricity parameter is 0.3 at the inlet of the article, 0.4 in a middle segment of the article, and 0.5 right before the outlet of the article, and the eccentricity parameter directly downstream of the outlet is 0.1, the eccentricity parameter directly downstream of the outlet is 80% lower than the highest eccentricity parameter (i.e., 0.5) at any segment of the article.

According to some embodiments, the article comprises one or more constricted regions (e.g., with one or more rotational flow generation features and/or obstructions), protrusions on an inner surface, ribs on an inner surface, and/or fins on an inner surface. In certain instances, the inclusion of one or more constricted regions (e.g., with one or more rotational flow generation features and/or obstructions), protrusions on an inner surface, ribs on an inner surface, and/or fins on an inner surface in the article maintains or lowers the eccentricity parameter directly downstream of the outlet compared to the highest eccentricity parameter at any segment of the article.

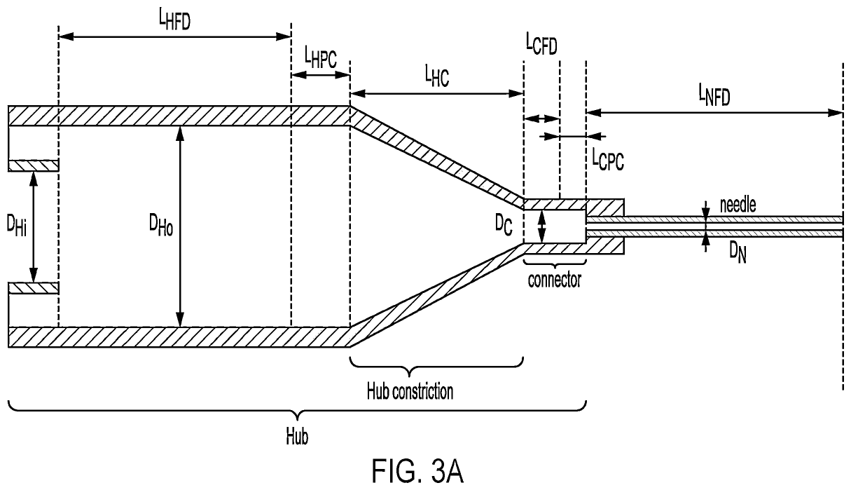

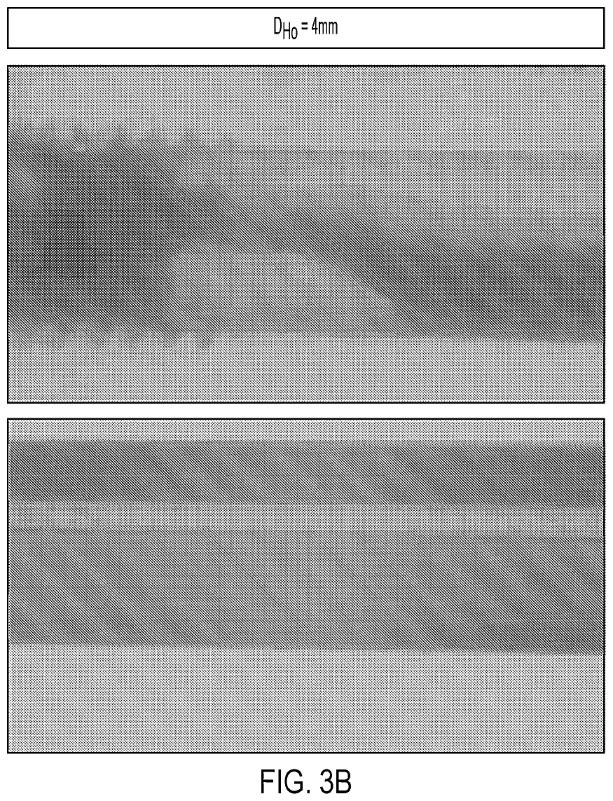

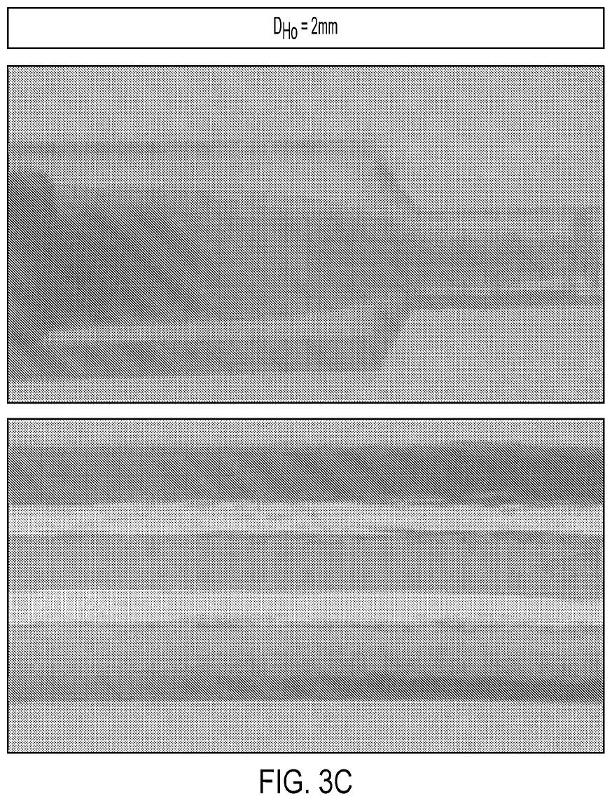

As used herein, a constricted region is a region with a smaller diameter than a region immediately upstream of that region. For example, in FIG. 3A, the tapered region is a constricted region as its diameter is smaller than the region immediately upstream of it.

According to certain embodiments, the timescale of convection is less than the timescale of eccentricity in the article. For example, in some embodiments, the timescale of convection is less than or equal to 90%, less than or equal to 80%, less than or equal to 70%, less than or equal to 60%, or less than or equal to 50% of the timescale of eccentricity in the article. In certain cases, the timescale of convection is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when the timescale of convection (T_{c}) is less than the timescale of eccentricity (tₑ), the fluids do not substantially exhibit eccentricity while in the system (e.g., the needle and/or article).

In certain embodiments, the difference in the density of the first fluid and the density of the second fluid is less than or equal to 400 kg/m³, less than or equal to 200 kg/m³, less than or equal to 100 kg/m³, or less than or equal to 50 kg/m³. In some embodiments, the difference in the density of the first fluid and the density of the second fluid is greater than or equal to 0 kg/m³, greater than or equal to 5 kg/m³, greater than or equal to 10 kg/m³, or greater than or equal to 25 kg/m³. Combinations of these ranges are also possible (e.g., greater than or equal to 0 kg/m³ and less than or equal to 400 kg/m³ or greater than or equal to 5 kg/m³ and less than or equal to 400 kg/m³).

In some embodiments, the article comprises one or more tapered regions. In certain instances, the external angle of one or more of the one or more tapered regions is less than or equal to 90 degrees, less than or equal to 80 degrees, less than or equal to 70 degrees, less than or equal to 60 degrees, less than or equal to 50 degrees, less than or equal to 40 degrees, or less than or equal to 30 degrees. In some cases, the external angle of one or more of the one or more tapered regions is greater than or equal to equal to 15 degrees, greater than or equal to equal to 20 degrees, greater than or equal to equal to 30 degrees, greater than or equal to equal to 40 degrees, greater than or equal to equal to 50 degrees, or greater than or equal to equal to 60 degrees. Combinations of these ranges are also possible (e.g., greater than or equal to 15 degrees and less than or equal to 90 degrees). For example, in FIG. 3A, in some embodiments, the tapered region (labeled “Hub constriction”) has an external angle α of less than 90 degrees.

In accordance with certain embodiments, the article has a ratio of L_{HPC}/D_{HO} of less than or equal to 2, where L_{HPC} is the pre-constriction flow length in the article (see, e.g., FIG. 3A) and D_{HO} is the largest inner diameter in the article (see, e.g., FIG. 3A). L_{HPC} may be determined visually by adding particles that can be visually distinguished (e.g., dye particles) into the fluid. The L_{HPC} region begins as the inner fluid (e.g., first fluid and/or fluid from the first conduit) begins curving inward.

In some embodiments, the article comprises a connector region. In certain instances, the connector region connects the remainder of the article to a needle. According to some embodiments, the article has a ratio of L_{CPC}/D_{c} of less than or equal to 2, wherein L_{CPC} is the pre-constriction flow length in a connector region (see, e.g., FIG. 3A) and D_{c} is the inner diameter of the connector region (see, e.g., FIG. 3A). L_{CPC}may be determined visually by adding particles that can be visually distinguished (e.g., dye particles) into the fluid. The L_{CPC} region begins as the inner fluid (e.g., first fluid and/or fluid from the first conduit) begins curving inward.

In certain embodiments, the article contains the first fluid and the second fluid. In some embodiments, the length (L) and diameter (D) of at least a portion of the article (e.g., at least a portion of an article having a length of at least 0.05 millimeters, at least 0.1 millimeters, at least 0.3 millimeters, at least 0.5 millimeters, or at least 1 millimeter) (e.g., at least a portion of an article with a uniform length and uniform diameter) satisfies the following equation for the first fluid and the second fluid:

$$\frac{L π {D}^{2}}{4 {Q}_{avg} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} < 1$$

where ρₒ is the density of the second fluid, ρᵢ is the density of the first fluid, μₒ is the viscosity of the second fluid, μᵢ is the viscosity of the first fluid, Q_{avg} is the average flowrate of the first fluid and the second fluid, L is the length of the portion of the article, θ is the angle between the length of the portion of the article and the horizontal plane, g is the gravitational constant, and D is the average diameter of the portion of the article.

In some embodiments, the length (L) and diameter (D) of at least a portion of the article (e.g., at least a portion of an article having a length of at least 0.05 millimeters, at least 0.1 millimeters, at least 0.3 millimeters, at least 0.5 millimeters, or at least 1 millimeter) (e.g., at least a portion of an article with a uniform length and uniform diameter) satisfies the following equation for the first fluid and the second fluid:

$$\frac{L π {D}^{2}}{4 {Q}_{total} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$

where ρₒ is the density of the second fluid, ρᵢ is the density of the first fluid, μₒ is the viscosity of the second fluid, μᵢ is the viscosity of the first fluid, Qₜₒₜₐₗ is the total flowrate of both fluids through the portion of the article, L is the length of the portion of the article, θ is the angle between the length of the portion of the article and the horizontal plane, g is the gravitational constant, and D is the average diameter of the portion of the article.

In some embodiments, the length (L) and diameter (D) of at least a portion of the article (e.g., at least a portion of an article having a length of at least 0.05 millimeters, at least 0.1 millimeters, at least 0.3 millimeters, at least 0.5 millimeters, or at least 1 millimeter) (e.g., at least a portion of an article with a uniform length and uniform diameter) satisfies the following equation for the first fluid and the second fluid:

$$\frac{LAi}{Qi \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$

where ρₒ is the density of the second fluid, ρᵢ is the density of the first fluid, μₒ is the viscosity of the second fluid, μᵢ is the viscosity of the first fluid, Qi is the flowrate of the inner fluid through the portion of the article, L is the length of the portion of the article, θ is the angle between the length of the portion of the article and the horizontal plane, g is the gravitational constant, and D is the average diameter of the portion of the article.

Ai may be estimated as shown below:

$${r}_{i}^{*} = \frac{{r}_{o}}{\sqrt{2 - {μ}_{o} / {μ}_{i}}} \qquad ( Equation 1 )$$

$${A}_{i} = {π ( {r}_{i}^{*} )}^{2} \qquad ( Equation 2 )$$

Where rᵢ* is the optimal radius of the inner fluid, μₒ is the dynamic viscosity of the outer fluid, μᵢ is the dynamic viscosity of the inner fluid, rₒ is the radius of the outer fluid, and Aᵢ is the cross-sectional area of the inner fluid as it flows through the portion of the article.

In some embodiments, at least a portion (e.g., at least 10%, at least 25%, at least 50%, at least 75%, at least 90%, or all) of the article comprises a biocompatible material.

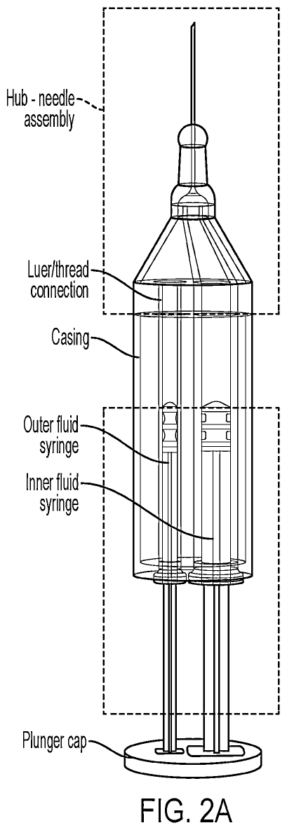

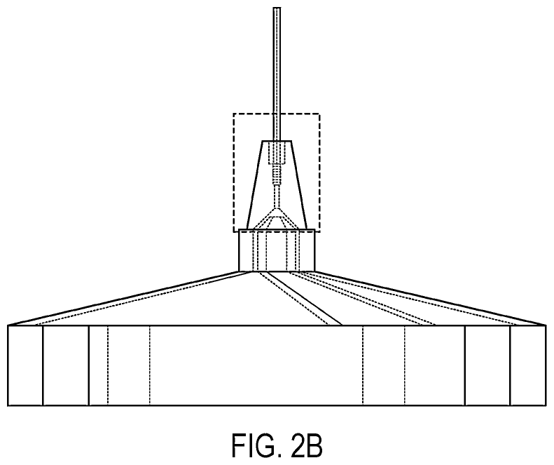

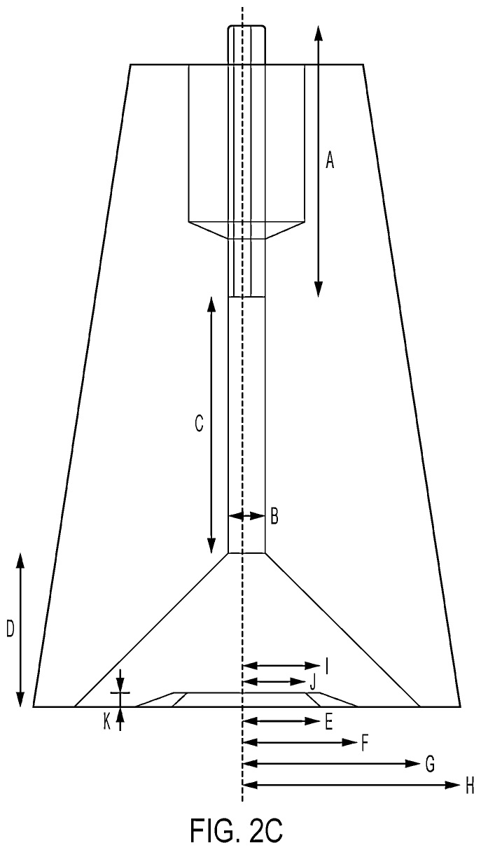

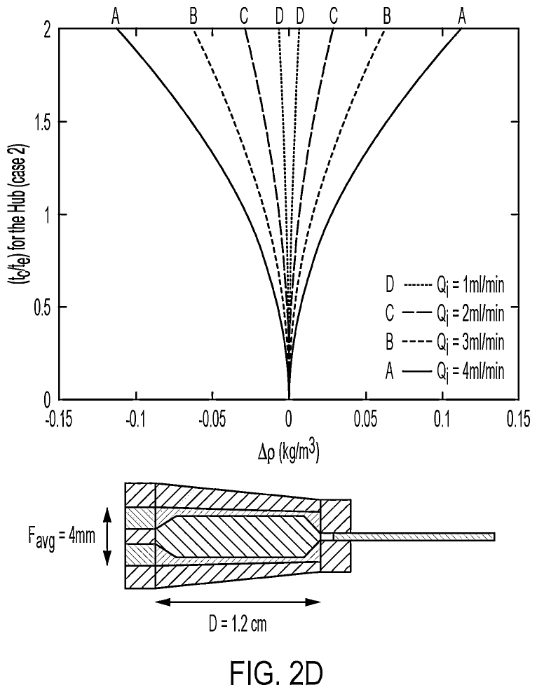

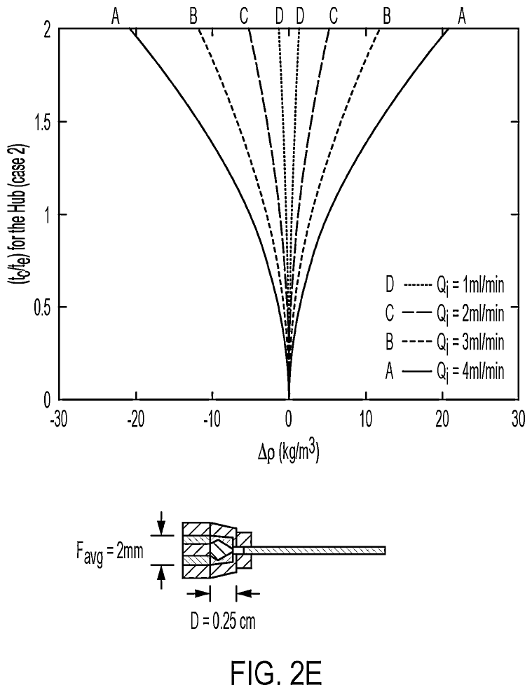

Systems (e.g., for delivery of a fluid) are also described herein. One such system is illustrated schematically in FIGS. 1C-1D and 2A-2C.

In some embodiments, the system comprises the article (e.g., any article disclosed herein). For example, in FIG. 1C, in certain instances, system 120 comprises article 100. Similarly, in FIG. 1D, in some cases, system 120 comprises article 100.

In certain embodiments, the system comprises a needle. For example, in FIG. 1D, in some embodiments, system 120 comprises needle 108. In some embodiments, the article is configured to be fluidically connected to a needle (e.g., the outlet of the article is configured to be fluidically connected to a needle). For example, in FIG. 1A, in some instances, article 100 is configured to be fluidically connected to a needle. In accordance with some embodiments, the needle is fluidically connected to the article (e.g., to the outlet of the article). For example, in FIG. 1D, in certain cases, needle 108 is fluidically connected to article 100.

According to some embodiments, the system comprises a first conduit and a second conduit. For example, in FIG. 1C, in some cases, system 120 comprises first conduit 106 and second conduit 107. Similarly, in FIG. 1D, in certain instances, system 120 comprises first conduit 106 and second conduit 107. In accordance with certain embodiments, the first conduit and second conduit are fluidically connected to the article (e.g., the inlet of the article). For example, in FIG. 1C, in some cases, first conduit 106 and second conduit 107 are fluidically connected to article 100. Similarly, in FIG. 1D, in certain instances, first conduit 106 and second conduit 107 are fluidically connected to article 100.

In accordance with some embodiments, the first conduit is arranged in a side-by-side configuration with the second conduit. For example, in FIG. 1D, in some instances, first conduit 106 is arranged in a side-by-side configuration with second conduit 107. In some cases, the first conduit comprises a longitudinal axis, the second conduit comprises a longitudinal axis, and at least a portion (e.g., at least 10%, at least 25%, at least 50%, at least 75%, at least 90%, or all) of the longitudinal axis of the first conduit is within 10 degrees (e.g., within 5 degrees or within 2 degrees) of parallel (or is parallel) to at least a portion (e.g., at least 10%, at least 25%, at least 50%, at least 75%, at least 90%, or all) of the longitudinal axis of the second conduit.

In certain embodiments, the system comprises a chamber. For example, in FIG. 1D, in some instances, system 120 comprises chamber 109. In some embodiments, the chamber comprises a first internal volume and a second internal volume. For example, in FIG. 1D, in certain cases, chamber 109 comprises first internal volume 110 and second internal volume 111. In accordance with certain embodiments, the first internal volume is fluidically connected to the first conduit and/or the inlet of the article. For example, in FIG. 1D, in some embodiments, first internal volume 110 is fluidically connected to first conduit 106 and/or the inlet of the article. In accordance with some embodiments, the second internal volume is fluidically connected to the second conduit and/or the inlet of the article. For example, in FIG. 1D, in certain embodiments, second internal volume 111 is fluidically connected to second conduit 107 and/or the inlet of the article.

In some embodiments, the second conduit axially surrounds the first conduit. For example, in FIG. 1C, in accordance with certain embodiments, second conduit 107 axially surrounds first conduit 106, similarly to how second fluid 105 axially surrounds first fluid 104 in FIG. 1B, in some cases.

According to certain embodiments, the system comprises a first plunger. For example, in FIG. 1D, in certain instances, system 120 comprises first plunger 112. In some cases, the first plunger is associated with (e.g., at least partially (e.g., at least 10%, at least 25%, at least 50%, at least 75%, at least 90%, or fully) disposed in) the first conduit. For example, in FIG. 1D, in some cases, first plunger 112 is associated with first conduit 106.

According to some embodiments, the system comprises a second plunger. For example, in FIG. 1D, in certain instances, system 120 comprises second plunger 113. In some cases, the second plunger is associated with (e.g., at least partially (e.g., at least 10%, at least 25%, at least 50%, at least 75%, at least 90%, or fully) disposed in) the second conduit. For example, in FIG. 1D, in some cases, second plunger 113 is associated with second conduit 107.

In accordance with some embodiments, the system comprises a solid body. For example, in FIG. 1D, in some instances, system 120 comprises solid body 114. In certain cases, the solid body connects the first plunger and the second plunger. For example, in FIG. 1D, in accordance with some embodiments, solid body 114 connects first plunger 112 and second plunger 113. As would be understood by those of ordinary skill in the art, a solid body is a body that includes a solid component. Solid bodies may, in certain cases, include cavities and/or be hollow, as long as other portions of the solid body are made of solid material. In some embodiments, the first plunger, the second plunger, and the solid body are all part of a single component made of the same material. In other embodiments, the first plunger, the second plunger, and/or the solid body can be made of different materials and assembled together. Other configurations are also possible.

In accordance with certain embodiments, the system is configured such that when the first plunger and the second plunger are compressed, fluid within the first conduit is transported to the article and fluid within the second conduit is transported to the article such that the fluid from the second conduit at least partially (e.g., partially or completely) axially surrounds fluid from the first conduit in the article. For example, in FIG. 1D, in some embodiments, system 120 is configured such that when first plunger 112 and second plunger 113 are compressed, fluid within first conduit 106 is transported to article 100 and fluid within second conduit 107 is transported to article 100, such that the fluid from second conduit 107 at least partially (e.g., partially or completely) axially surrounds fluid from first conduit 106 in article 100.

In some embodiments, the system is configured such that when the first plunger and the second plunger are compressed, fluid within the first conduit is transported to the needle and fluid within the second conduit is transported to the needle such that the fluid from the second conduit at least partially (e.g., partially or completely) axially surrounds fluid from the first conduit in the needle. For example, in FIG. 1D, in certain embodiments, system 120 is configured such that when first plunger 112 and second plunger 113 are compressed, fluid within first conduit 106 is transported to needle 108 and fluid within second conduit 107 is transported to needle 108, such that the fluid from second conduit 107 at least partially (e.g., partially or completely) axially surrounds fluid from first conduit 106 in needle 108.

Methods (e.g., for delivery of a fluid) are described herein. In some embodiments, the method comprises delivering one or more fluids using an article and/or system (e.g., disclosed herein).

According to some embodiments, the outer fluid (e.g., second fluid and/or fluid from the second conduit) preferentially wets an interior surface of the needle and/or the article relative to the inner fluid (e.g., first fluid and/or fluid from the first conduit).

In some embodiments, the outer fluid preferentially wets an interior surface of the needle and/or article relative to the inner fluid when for the inner fluid, the outer fluid, and the interior surface of the needle and/or article, the spreading coefficient (Sₒₙ₍ᵢ₎) is greater than or equal to 0. FIG. 6 is a schematic illustration of a droplet of the outer fluid on the interior surface of the needle and/or article, where the outer droplet is surrounded by the inner fluid. The spreading coefficient can be determined according to the following equations:

$${S}_{on ( i )} = {γ}_{ni} - ( {γ}_{no} + {γ}_{oi} ) \qquad ( Equation 3 )$$

$$cos ( {θ}_{on ( i )} ) = \frac{{γ}_{ni} - {γ}_{no}}{{γ}_{oi}} \qquad ( Equation 4 )$$

$${S}_{on ( i )} = {γ}_{oi} ( cos ( {θ}_{on ( i )} ) - 1 ) \qquad ( Equation 5 )$$

In the equations above, gamma (γ) is the surface tensions of the various interfaces involved, where n is the subscript for an interior surface of the needle and/or article, o is the subscript for the outer fluid, and i is the subscript for the inner fluid. For example, [symbol] ₙᵢ denotes the surface tension between the needle (and/or article) and the inner fluid, [symbol] ₙₒ denotes the surface tension between the needle (and/or article) and the outer fluid, and [symbol] ₒᵢ denotes the surface tension between the outer fluid and the inner fluid. For example, in some embodiments, cos(θₒₙ₍ᵢ₎) and [symbol] ₒᵢ are measured, and the spreading coefficient is determined by Equation 5. The spreading coefficient is specific to the three components (e.g., the interior surface of the needle and/or article, the inner fluid, and the outer fluid).

In certain embodiments, the inner fluid (e.g., first fluid and/or fluid from the first conduit) does not contact an interior surface of the needle and/or article. According to some embodiments, the inner fluid does not contact an interior surface of the needle and/or article for a period of time. For example, in some cases, the period of time is between initiating flow of the inner fluid and/or outer fluid and ejection of the inner fluid and/or outer fluid from the needle and/or article. In certain cases, the period of time is at least a portion of time (e.g., at least 50%, at least 75%, at least 90%, or the entirety of the time) between initiating flow of the fluid and ejection of fluid from the needle and/or article.

According to some embodiments, the inner fluid (e.g., first fluid and/or fluid from the first conduit) comprises a drug, a monoclonal antibody, an enzyme, a peptide, a recombinant therapeutic protein, a biologic, a bone putty, a hydrogel, cells, and/or a biopharmaceutical. For example, in certain embodiments, the inner fluid comprises a concentrated drug formulation (e.g., biologic).

According to certain embodiments, the outer fluid (e.g., second fluid and/or fluid from the second conduit) has a lower viscosity than the inner fluid. In some embodiments, the ratio of the viscosity of the inner fluid to the viscosity of the outer fluid (μᵢ/μₒ)>1. In some embodiments, the ratio of the viscosity of the inner fluid to the viscosity of the outer fluid (μᵢ/μₒ) is greater than or equal to 3, greater than or equal to 5, greater than or equal to 8, or greater than or equal to 10.

In some cases, the outer fluid (e.g., second fluid and/or fluid from the second conduit) comprises water, a buffer (e.g., a pharmaceutically acceptable buffer, such as a buffer used in a pharmaceutical product, such as a biologic), a formulation (e.g., a pharmaceutical formulation, such as a biologic formulation), a water-based solution, saline, a biocompatible oil (e.g., squalene, a fluorinated oil (e.g., HFE-7500), mineral oil, and/or triglyceride oil), benzyl benzoate, a metabolizable oil, an immunologic adjuvant (e.g., MF59, AS02, AS03 and/or AS04), and/or safflower oil.

In some embodiments, the outer fluid (e.g., second fluid and/or fluid from the second conduit) and inner fluid (e.g., first fluid and/or fluid from the first conduit) are immiscible. For example, according to certain embodiments, neither the outer fluid nor the inner fluid is soluble in the other in an amount of more than 0.001 mass fraction, more than 0.0001 mass fraction, or more than 0.00001 mass fraction. In certain embodiments, the outer fluid and inner fluid are immiscible at the temperature at which the fluids are flowed. In some cases, the outer fluid and inner fluid are immiscible at 25° C.

The use of immiscible inner fluids (e.g., first fluid and/or fluid from the first conduit) and outer fluids (e.g., second fluid and/or fluid from the second conduit) is not necessarily required, and in some embodiments, the outer fluid and inner fluid are miscible. For example, according to some embodiments, the outer fluid and/or the inner fluid is soluble in the other in an amount of more than 0.001 mass fraction, more than 0.01 mass fraction, or more than 0.1 mass fraction. In certain embodiments, the outer fluid and inner fluid are miscible at the temperature at which the fluids are flowed. In some cases, the outer fluid and inner fluid are miscible at 25° C.

For the systems, articles, and methods described herein, the timescale of convection (T_{c}) is how long the inner fluid (e.g., first fluid and/or fluid from the first conduit) and outer fluid (e.g., second fluid and/or fluid from the second conduit) take to travel through the system (e.g., the needle, the chamber, and/or the article) while they are in direct contact with each other.

In some embodiments, the timescale of convection may be approximated by estimating the average volumetric flow rate of the multi-fluid system. In certain embodiments, the average volumetric flowrate and timescale of convection may be approximated using the following equations:

$${Q}_{avg} = \frac{{Q}_{i} + {Q}_{o}}{2} \qquad ( Equation 6 )$$

$${T}_{c} = \frac{L}{\overline{V}} = \frac{L {A}_{c}}{{Q}_{avg}} \qquad ( Equation 7 )$$

Where Q_{avg} is the average flowrate of the inner and outer fluids, Qi is the volumetric flowrate of the inner fluid, Qo is the volumetric flowrate of the outer fluid, L is the length of the system, A_{c} is the cross-sectional area of the system, and V̅ is the average linear velocity.

According to certain embodiments,

$$\frac{L {A}_{c}}{{Q}_{avg}}$$

is less than the timescale of eccentricity in the article. For example, in some embodiments,

$$\frac{L {A}_{c}}{{Q}_{avg}}$$

is less than or equal to 90%, less than or equal to 80%, less than or equal to 70%, less than or equal to 60%, or less than or equal to 50% of the timescale of eccentricity in the article. In certain cases,

$$\frac{L {A}_{c}}{{Q}_{avg}}$$

is greater than or equal to 10%, greater than or equal to 20%, greater than or equal to 30%, or greater than or equal to 40% of the timescale of eccentricity in the article.
Combinations of these ranges are also possible (e.g., greater than or equal to 10% and less than or equal to 90%). In some embodiments, when

$$\frac{L {A}_{c}}{{Q}_{avg}}$$

is less than the timescale of eccentricity (tₑ), the fluids do not substantially exhibit eccentricity while in the system (e.g., the needle and/or article).

In certain embodiments,

$$\frac{L {A}_{c}}{{Q}_{avg}}$$

is less than the timescale of mixing (tₘ) in one or more portions of the system (e.g., in the needle and/or in the article) or in the entire system. For example, in some embodiments,

$$\frac{L {A}_{c}}{{Q}_{avg}}$$

is less than the timescale of mixing in the needle and/or

$$\frac{L {A}_{c}}{{Q}_{avg}}$$

is less than the timescale of mixing in the article. For example, in some embodiments, the ratio of

$$\frac{L {A}_{c}}{{Q}_{avg}}$$

for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when

$$\frac{L {A}_{c}}{{Q}_{avg}}$$

is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the

$$\frac{L {A}_{c}}{{Q}_{avg}}$$

and/or the ratio of

$$\frac{L {A}_{c}}{{Q}_{avg}}$$

to the timescale of eccentricity. For example, in accordance with certain embodiments, a ratio of

$$\frac{L {A}_{c}}{{Q}_{avg}}$$

to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of

$$\frac{L {A}_{c}}{{Q}_{avg}}$$

to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\frac{L {A}_{c}}{{Q}_{avg}}$$

is less than

$$\sqrt{\frac{2 s}{\vert g cos ( θ ) ( 1 - \frac{{ρ}_{i}}{{ρ}_{o}} ) \vert}} .$$

For example, in some embodiments

$$\frac{L {A}_{c}}{{Q}_{avg}}$$

is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{0}} ) \vert}}$$

in the article. In certain cases,

$$\frac{L {A}_{c}}{{Q}_{a ν g}}$$

is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{0}} ) \vert}}$$

in the article. Combinations of these ranges are also possible (e.g., greater than or equal to 10% and less than or equal to 90%). In some embodiments, when

$$\frac{L {A}_{c}}{{Q}_{a ν g}}$$

is less than

$$\sqrt{\frac{2 s}{\vert g cos ( θ ) ( 1 - \frac{{ρ}_{i}}{{ρ}_{0}} ) \vert}} ,$$

the fluids do not substantially exhibit eccentricity while in the system (e.g., the needle and/or article).

In certain embodiments,

$$\frac{L {A}_{c}}{{Q}_{a ν g}}$$

is less than

$$\frac{{l}_{d}^{2}}{D i}$$

in one or more portions of the system (e.g., in the needle and/or in the article) or in the entire system. For example, in some embodiments,

$$\frac{L {A}_{c}}{{Q}_{a ν g}}$$

is less than

$$\frac{{l}_{d}^{2}}{D i}$$

in the needle and/or

$$\frac{L {A}_{c}}{{Q}_{a ν g}}$$

is less than

$$\frac{{l}_{d}^{2}}{D i}$$

in the article. For example, in some embodiments, the ratio of

$$\frac{L {A}_{c}}{{Q}_{a ν g}}$$

for the inner fluid and outer fluid to

$$\frac{{l}_{d}^{2}}{D i}$$

for the inner fluid and outer fluid is less than or equal to 1, less than or equal to 0.75, less than or equal to 0.5, less than or equal to 0.1 or less than or equal to 0.01. In some embodiments, when

$$\frac{{LA}_{c}}{{Q}_{avg}}$$

is less than

$$\frac{{l}_{d}^{2}}{Di} ,$$

the fluids do not substantially mix while in the system or a portion thereof (e.g., the needle and/or article).

In some embodiments, the densities of the inner and outer fluids and/or the volumetric flow rate (Q) affects

$$\frac{{LA}_{c}}{{Q}_{avg}}$$

and/or the ratio of

$$\frac{{LA}_{c}}{{Q}_{avg}}$$

to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{avg}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{c}}{{Q}_{avg}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\frac{{LA}_{c}}{{Q}_{avg}}$$

is less than

$$\sqrt{\frac{2 s}{\vert g cos ( θ ) ( 1 - \frac{{ρ}_{o}}{{ρ}_{i}} ) \vert}} .$$

For example, in some embodiments

$$\frac{{LA}_{c}}{{Q}_{avg}}$$

is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases,

$$\frac{{LA}_{c}}{{Q}_{avg}}$$

is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when

$$\frac{L {A}_{c}}{{Q}_{a ν g}}$$

is less than

$$\sqrt{\frac{2 s}{\vert g cos ( θ ) ( 1 - \frac{{ρ}_{o}}{{ρ}_{i}} ) \vert}} ,$$

the fluids do not substantially exhibit eccentricity while in the system (e.g., the needle and/or article).

In some embodiments, the densities of the inner and outer fluids and/or the volumetric flow rate (Q) affects

$$\frac{L {A}_{c}}{{Q}_{a ν g}}$$

and/or the ratio of

$$\frac{L {A}_{c}}{{Q}_{a ν g}}$$

to

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{L {A}_{c}}{{Q}_{a ν g}}$$

to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{L {A}_{c}}{{Q}_{a ν g}}$$

to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In some embodiments, the timescale of convection may be approximated by estimating the total volumetric flow rate of the multi-fluid system. In certain embodiments, the total volumetric flowrate and timescale of convection may be approximated using the following equations:

$${Q}_{total} = {Q}_{i} + {Q}_{o} \qquad ( Equation 8 )$$

$${T}_{c} = \frac{L}{\overline{V}} = \frac{L {A}_{c}}{{Q}_{total}} \qquad ( Equation 9 )$$

Where Qₜₒₜₐₗ is the total volumetric flowrate of the inner and outer fluids, Qi is the volumetric flowrate of the inner fluid, Qo is the volumetric flowrate of the outer fluid, L is the length of the system, A_{c} is the cross-sectional area of the system, and V̅ is the average linear velocity.

According to certain embodiments,

$$\frac{L {A}_{c}}{{Q}_{total}}$$

is less than the timescale of eccentricity in the article. For example, in some embodiments,

$$\frac{L {A}_{c}}{{Q}_{total}}$$

is less than or equal to 90%, less than or equal to 80%, less than or equal to 70%, less than or equal to 60%, or less than or equal to 50% of the timescale of eccentricity in the article. In certain cases,

$$\frac{L {A}_{c}}{{Q}_{total}}$$

is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when

$$\\ \frac{{LA}_{c}}{{Q}_{total}}$$

is less than the timescale of eccentricity (tₑ), the fluids do not substantially exhibit eccentricity while in the system (e.g., the needle and/or article).

In certain embodiments,

$$\\ \frac{{LA}_{c}}{{Q}_{total}}$$

is less than the timescale of mixing (tₘ) in one or more portions of the system (e.g., in the needle and/or in the article) or in the entire system. For example, in some embodiments,

$$\\ \frac{{LA}_{c}}{{Q}_{total}}$$

is less than the timescale of mixing in the needle and/or

$$\\ \frac{{LA}_{c}}{{Q}_{total}}$$

is less than the timescale of mixing in the article. For example, in some embodiments, the ratio of

$$\\ \frac{{LA}_{c}}{{Q}_{total}}$$

for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when

$$\\ \frac{{LA}_{c}}{{Q}_{total}}$$

is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the

$$\\ \frac{{LA}_{c}}{{Q}_{total}}$$

and/or the ratio of

$$\\ \frac{{LA}_{c}}{{Q}_{total}}$$

to the timescale of eccentricity. For example, in accordance with certain embodiments, a ratio of

$$\\ \frac{{LA}_{c}}{{Q}_{total}}$$

to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of

$$\\ \frac{{LA}_{c}}{{Q}_{total}}$$

to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\\ \frac{{LA}_{c}}{{Q}_{total}}$$

is less than

$$\\ \sqrt{\frac{2 s}{\vert g cos ( θ ) ( 1 - \frac{{ρ}_{i}}{{ρ}_{o}} ) \vert}} .$$

For example, in some embodiments

$$\\ \frac{{LA}_{c}}{{Q}_{total}}$$

is less than or equal to 90%, less than or equal to 80%, less than or equal to 70%, less than or equal to 60%, or less than or equal to 50% of

$$\\ \sqrt{\frac{2 s}{\vert g cos ( θ ) ( 1 - \frac{{ρ}_{i}}{{ρ}_{o}} ) \vert}}$$

in the article. In certain cases,

$$\\ \frac{{LA}_{c}}{{Q}_{total}}$$

is greater than or equal to 10%, greater than or equal to 20%, greater than or equal to 30%, or greater than or equal to 40% of

$$\\ \sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when

$$\\ \frac{{LA}_{c}}{{Q}_{total}}$$

is less than

$$\\ \sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In certain embodiments,

$$\\ \frac{{LA}_{c}}{{Q}_{total}}$$

is less than

$$\\ \frac{{l}_{d}^{{}_{} 2}}{Di}$$

in one or more portions of the system (e.g., in the needle and/or in the article) or in the entire system. For example, in some embodiments,

$$\\ \frac{{LA}_{c}}{{Q}_{total}}$$

is less than

$$\\ \frac{{l}_{d}^{{}_{} 2}}{Di}$$

in the needle and/or

$$\\ \frac{{LA}_{c}}{{Q}_{total}}$$

is less than

$$\\ \frac{{l}_{d}^{{}_{} 2}}{Di}$$

in the article. For example, in some embodiments, the ratio of

$$\\ \frac{{LA}_{c}}{{Q}_{total}}$$

for the inner fluid and outer fluid to

$$\\ \frac{{l}_{d}^{{}_{} 2}}{Di}$$

for the inner fluid and outer fluid is less than or equal to 1, less than or equal to 0.75, less than or equal to 0.5, less than or equal to 0.1 or less than or equal to 0.01. In some embodiments, when

$$\\ \frac{{LA}_{c}}{{Q}_{total}}$$

is less than

$$\\ \frac{{l}_{d}^{{}_{} 2}}{Di} ,$$

the fluids do not substantially mix while in the system or a portion thereof (e.g., the needle and/or article).

In some embodiments, the densities of the inner and outer fluids and/or the volumetric flow rate (Q) affects

$$\\ \frac{{LA}_{c}}{{Q}_{total}}$$

and/or the ratio of

$$\\ \frac{{LA}_{c}}{{Q}_{total}}$$

to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{L {A}_{c}}{{Q}_{t o t a l}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{L {A}_{c}}{{Q}_{t o t a l}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\frac{L {A}_{c}}{{Q}_{t o t a l}}$$

is less than

$$\sqrt{\frac{2 s}{\vert g cos ( θ ) ( 1 - \frac{{ρ}_{o}}{{ρ}_{i}} ) \vert}} .$$

For example, in some embodiments

$$\frac{L {A}_{c}}{{Q}_{t o t a l}}$$

is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}} .$$

in the article. In certain cases,

$$\frac{L {A}_{c}}{{Q}_{t o t a l}}$$

is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when

$$\frac{L {A}_{c}}{{Q}_{t o t a l}}$$

is less than

$$\sqrt{\frac{2 s}{\vert g cos ( θ ) ( 1 - \frac{{ρ}_{o}}{{ρ}_{i}} ) \vert}} .$$

the fluids do not substantially exhibit eccentricity while in the system (e.g., the needle and/or article).

In some embodiments, the densities of the inner and outer fluids and/or the volumetric flow rate (Q) affects

$$\frac{L {A}_{c}}{{Q}_{t o t a l}}$$

and/or the ratio of

$$\frac{L {A}_{c}}{{Q}_{t o t a l}}$$

to

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{L {A}_{c}}{{Q}_{t o t a l}}$$

to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{L {A}_{c}}{{Q}_{t o t a l}}$$

to

$$\sqrt{\frac{2 s}{\vert g cos ( θ ) ( 1 - \frac{{ρ}_{o}}{{ρ}_{i}} ) \vert}} .$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In some embodiments, the timescale of convection may be approximated using the following equations:

$${T}_{c} = \frac{L}{\overline{V}} = \frac{L A i}{{Q}_{t}} \qquad ( Equation 10 )$$

where Ai may be estimated as shown below:

$${r}_{i}^{*} = \frac{{r}_{o}}{\sqrt{2 - {μ}_{0} / {μ}_{i}}} \qquad ( Equation 11 )$$

$${A}_{i} = {π ( {r}_{i}^{*} )}^{2} \qquad ( Equation 12 )$$

Where rᵢ* is the optimal radius of the inner fluid, μₒ is the dynamic viscosity of the outer fluid, μᵢ is the dynamic viscosity of the inner fluid, rₒ is the radius of the outer fluid, L is the length of the system, and Aᵢ is the cross-sectional area of the inner fluid as it flows through a region of interest of the system, and V̅ is the average linear velocity.

According to certain embodiments,

$$\frac{L {A}_{i}}{{Q}_{i}}$$

is less than the timescale of eccentricity in the article. For example, in some embodiments,

$$\frac{L {A}_{i}}{{Q}_{i}}$$

is less than or equal to 90%, less than or equal to 80%, less than or equal to 70%, less than or equal to 60%, or less than or equal to 50% of the timescale of eccentricity in the article. In certain cases,

$$\frac{L {A}_{i}}{{Q}_{i}}$$

is greater than or equal to 10%, greater than or equal to 20%, greater than or equal to 30%, or greater than or equal to 40% of the timescale of eccentricity in the article. Combinations of these ranges are also possible (e.g., greater than or equal to 10% and less than or equal to 90%). In some embodiments, when

$$\frac{L {A}_{i}}{{Q}_{i}}$$

is less than the timescale of eccentricity (tₑ), the fluids do not substantially exhibit eccentricity while in the system (e.g., the needle and/or article).

In certain embodiments,

$$\frac{L {A}_{i}}{{Q}_{i}}$$

is less than the timescale of mixing (tₘ) in one or more portions of the system (e.g., in the needle and/or in the article) or in the entire system. For example, in some embodiments,

$$\frac{L {A}_{i}}{{Q}_{i}}$$

is less than the timescale of mixing in the needle and/or

$$\frac{{LA}_{i}}{{Q}_{i}}$$

is less than the timescale of mixing in the article. For example, in some embodiments, the ratio of

$$\frac{{LA}_{i}}{{Q}_{i}}$$

for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when

$$\frac{{LA}_{i}}{{Q}_{i}}$$

is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the

$$\frac{{LA}_{i}}{{Q}_{i}}$$

and/or the ratio of

$$\frac{{LA}_{i}}{{Q}_{i}}$$

to the timescale of eccentricity. For example, in accordance with certain embodiments, a ratio of

$$\frac{{LA}_{i}}{{Q}_{i}}$$

to the timescale of eccentricity of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects the timescale of eccentricity. For example, in accordance with certain embodiments, the ratio of

$$\frac{{LA}_{i}}{{Q}_{i}}$$

to timescale of eccentricity of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\frac{{LA}_{i}}{{Q}_{i}}$$

is less than

$$\sqrt{\frac{2 s}{\vert g cos ( θ ) ( 1 - \frac{{ρ}_{i}}{{ρ}_{o}} ) \vert}} .$$

For example, in some embodiments

$$\frac{{LA}_{i}}{{Q}_{i}}$$

is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases,

$$\frac{{LA}_{i}}{{Q}_{i}}$$

is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when

$$\frac{{LA}_{i}}{{Q}_{i}}$$

is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In certain embodiments,

$$\frac{{LA}_{i}}{{Q}_{i}}$$

is less than

$$\frac{{l}_{d}^{2}}{Di}$$

in one or more portions of the system (e.g., in the needle and/or in the article) or in the entire system. For example, in some embodiments,

$$\frac{{LA}_{i}}{{Q}_{i}}$$

is less than

$$\frac{{l}_{d}^{2}}{Di}$$

in the needle and/or

$$\frac{{LA}_{i}}{{Q}_{i}}$$

is less than

$$\frac{{l}_{d}^{2}}{Di}$$

in the article. For example, in some embodiments, the ratio of

$$\frac{{LA}_{i}}{{Q}_{i}}$$

for the inner fluid and outer fluid to

$$\frac{{l}_{d}^{2}}{Di}$$

for the inner fluid and outer fluid is less than or equal to 1, less than or equal to 0.75, less than or equal to 0.5, less than or equal to 0.1 or less than or equal to 0.01. In some embodiments, when

$$\frac{{LA}_{i}}{{Q}_{i}}$$

is less than

$$\frac{{l}_{d}^{2}}{Di} ,$$

the fluids do not substantially mix while in the system or a portion thereof (e.g., the needle and/or article).

In some embodiments, the densities of the inner and outer fluids and/or the volumetric flow rate (Q) affects

$$\frac{{LA}_{i}}{{Q}_{i}}$$

and/or the ratio of

$$\frac{{LA}_{i}}{{Q}_{i}}$$

to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{i}}{{Q}_{i}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{i}}{{Q}_{i}}$$

to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

According to certain embodiments,

$$\frac{{LA}_{i}}{{Q}_{i}}$$

is less than

$$\sqrt{\frac{2 s}{\vert g cos ( θ ) ( 1 - \frac{{ρ}_{i}}{{ρ}_{o}} ) \vert}} .$$

For example, in some embodiments

$$\frac{{LA}_{i}}{{Q}_{i}}$$

is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases,

$$\frac{{LA}_{i}}{{Q}_{i}}$$

is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when

$$\frac{{LA}_{i}}{{Q}_{i}}$$

is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In some embodiments, the densities of the inner and outer fluids and/or the volumetric flow rate (Q) affects

$$\frac{{LA}_{i}}{{Q}_{i}}$$

and/or the ratio of

$$\frac{{LA}_{i}}{{Q}_{i}}$$

to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of

$$\frac{{LA}_{i}}{{Q}_{i}}$$

to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of

$$\frac{{LA}_{i}}{{Q}_{i}}$$

to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

For the systems and methods described herein, the timescale of eccentricity (tₑ) is the time for spatially stable eccentricity to arise in any part of the system (e.g., the needle, the chamber, and/or the article) comprising the inner fluid (e.g., first fluid and/or fluid from the first conduit) and outer fluid (e.g., second fluid and/or fluid from the second conduit).

In some embodiments, timescale of eccentricity may be approximated according to the following equation:

$${t}_{e} = \sqrt{\frac{2 s}{\vert g cos ( θ ) ( 1 - \frac{{ρ}_{i}}{{ρ}_{o}} ) \vert}} \qquad ( Equation 13 )$$

Where θ is the angle between the length of the needle and the horizontal plane, ρᵢ is density of the inner fluid, g is the gravitational constant and s is the displacement parameter (radial displacement of the centerline of the inner fluid from the axial centerline of the device), and ρₒ is density of the outer fluid.

According to certain embodiments, the timescale of convection is less than

$$\sqrt{\frac{2 s}{\vert g cos ( θ ) ( 1 - \frac{{ρ}_{i}}{{ρ}_{o}} ) \vert}}$$

in the article. For example, in some embodiments, the timescale of convection is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{i}}{{ρ}_{o}} ) \vert}}$$

in the article. In certain cases, the timescale of convection is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{i}}{{ρ}_{o}} ) \vert}}$$

in the article. Combinations of these ranges are also possible (e.g., greater than or equal to 10% and less than or equal to 90%). In some embodiments, when the timescale of convection (T_{c}) is less than

$$\sqrt{\frac{2 s}{\vert g cos ( θ ) ( 1 - \frac{{ρ}_{i}}{{ρ}_{o}} ) \vert}} ,$$

the fluids do not substantially exhibit eccentricity while in the system (e.g., the needle and/or article).

In some embodiments, the densities of the inner and outer fluids and/or the volumetric flow rate (Q) affects the timescale of convection and/or the ratio of the timescale of convection to

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of timescale of convection to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{i}}{{ρ}_{o}} ) \vert}} .$$

For example, in accordance with certain embodiments, a ratio of timescale of convection to

$$\sqrt{\frac{2 s}{\vert g cos ( θ ) ( 1 - \frac{{ρ}_{i}}{{ρ}_{o}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

In certain embodiments, timescale of eccentricity may be approximated according to the following equation:

$${t}_{e} = \sqrt{\frac{2 s}{\vert g cos ( θ ) ( 1 - \frac{{ρ}_{o}}{{ρ}_{i}} ) \vert}} \qquad ( Equation 14 )$$

Where θ is the angle between the length of the needle and the horizontal plane, ρᵢ is density of the inner fluid, g is the gravitational constant and s is the displacement parameter (radial displacement of the centerline of the inner fluid from the axial centerline of the device), and ρₒ is density of the outer fluid.

According to certain embodiments, the timescale of convection is less than

$$\sqrt{\frac{2 s}{\vert g cos ( θ ) ( 1 - \frac{{ρ}_{o}}{{ρ}_{i}} ) \vert}}$$

in the article. For example, in some embodiments, the timescale of convection is less than or equal to 90%, less than or equal to 80%, less than or equal to 70%, less than or equal to 60%, or less than or equal to 50% of

$$\sqrt{\frac{2 s}{\vert g cos ( θ ) ( 1 - \frac{{ρ}_{o}}{{ρ}_{i}} ) \vert}}$$

in the article. In certain cases, the timescale of convection is greater than or equal to 10%, greater than or equal to 20%, greater than or equal to 30%, or greater than or equal to 40% of

$$\sqrt{\frac{2 s}{\vert g cos ( θ ) ( 1 - \frac{{ρ}_{o}}{{ρ}_{i}} ) \vert}}$$

in the article. Combinations of these ranges are also possible (e.g., greater than or equal to 10% and less than or equal to 90%). In some embodiments, when the timescale of convection (T_{c}) is less than

$$\sqrt{\frac{2 s}{\vert g cos ( θ ) ( 1 - \frac{{ρ}_{o}}{{ρ}_{i}} ) \vert}} ,$$

the fluids do not substantially exhibit eccentricity while in the system (e.g., the needle and/or article).

In some embodiments, the densities of the inner and outer fluids and/or the volumetric flow rate (Q) affects the timescale of convection and/or the ratio of the timescale of convection to

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of timescale of convection to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with smaller differences in density between the inner fluid and outer fluid and/or with a higher average volumetric flow rate of the inner fluid (Qᵢ).

In some embodiments, the orientation of the system (e.g., the needle and/or article) affects

$$\sqrt{\frac{2 s}{\vert g cos ( θ ) ( 1 - \frac{{ρ}_{o}}{{ρ}_{i}} ) \vert}} .$$

For example, in accordance with certain embodiments, a ratio of timescale of convection to

$$\sqrt{\frac{2 s}{\vert g cos ( θ ) ( 1 - \frac{{ρ}_{o}}{{ρ}_{i}} ) \vert}}$$

of less than or equal to 1 is easier to achieve with the system (e.g., the needle and/or article) closer to vertical (90° from a line perpendicular to gravity), and more difficult to achieve closer to horizontal (0° from a line perpendicular to gravity).

The displacement parameter will, in some cases, depend on the flow rate ratio of the outer fluid (e.g., second fluid and/or fluid from the second conduit) and inner fluid (e.g., first fluid and/or fluid from the first conduit) (Qₒ/Qᵢ) and the inner diameter of the section of interest. The displacement length may be defined as (e.g., in the case of concentric core annular flow designed to minimize the pressure drop for transport of a viscous inner fluid through a needle) the distance required to reach a fully eccentric flow and can be written as:

$$s = \frac{D}{2} [ 1 - \frac{1}{\sqrt{2 - \frac{{μ}_{o}}{{μ}_{i}}}} ]$$

where D is the diameter of the section of interest where the two fluids are in contact with each other, μₒ is the viscosity of the outer fluid (lubricant), and μᵢ is the viscosity of the inner fluid (drug formulation).

For the systems, articles, and methods described herein, the timescale of mixing (tₘ) is the time needed for 50% of the outer fluid (e.g., second fluid and/or fluid from the second conduit) to mix with the inner fluid (e.g., first fluid and/or fluid from the first conduit) as they travel through the system or a portion thereof (e.g., the needle and/or the article) while they are in direct contact with each other. The timescale of mixing may be calculated using the following equation:

$${t}_{m} = \frac{{l}_{d}^{2}}{Di} \qquad ( Equation 15 )$$

Where Dᵢ is the diffusion coefficient of one or more components of the inner fluid (e.g., a drug (e.g., a biologic) in the inner fluid) in the outer fluid and l_{d} is the diameter of the part of the system (e.g., the needle and/or the article) where the fluids are in direct contact with each other. In embodiments where the system has portions with different diameters (e.g., a system comprising an article and a needle where the article has a larger diameter than the needle), the timescale of mixing may be determined using Equation 15 for each portion individually. In embodiments where the system has a varying geometry (e.g., if the article had an oval shape), the timescale of mixing may be determined using Equation 15 in conjunction with an integral approach.

In certain embodiments, the timescale of convection (T_{c}) is less than the timescale of mixing (tₘ) in one or more portions of the system (e.g., in the needle and/or in the article) or in the entire system. For example, in some embodiments, the timescale of convection is less than the timescale of mixing in the needle and/or the timescale of convection is less than the timescale of mixing in the article. For example, in some embodiments, the ratio of the timescale of convection (T_{c}) for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when the timescale of convection (T_{c}) is less than the timescale of mixing (tₘ), the fluids do not substantially mix while in the system or a portion thereof (e.g., the needle and/or article).

In some embodiments, the densities of the inner and outer fluids and/or the volumetric flow rate (Q) affects the timescale of convection and/or the ratio of the timescale of convection to the timescale of eccentricity. For example, in accordance with certain embodiments, a T_{c}/tₑ of less than or equal to 1 is easier to achieve with smaller differences in density between the inner fluid and outer fluid and/or with a higher average volumetric flow rate of the inner fluid (Qᵢ).

In certain embodiments, when the outer fluid flow rate is too low compared to the inner fluid flow rate, a viscous displacement regime is observed rather than an axially lubricated flow regime. In a viscous displacement regime, the outer fluid fills the entire cross-section of the needle and forces both the inner fluid and the outer fluid to back-flow into the outer fluid inlet. However, in certain cases, the backflow cannot be sustained due to the constant mass flux that is imposed on the outer fluid, resulting in a sudden overflow of the outer fluid into the needle. In some instances, this flow decreases until it is completely hindered once again, and the process repeats.

In accordance with some embodiments, the ratio of the volumetric flow rate of the outer fluid (Qₒ) (e.g., second fluid and/or fluid from the second conduit) to the volumetric flow rate of the inner fluid (Qᵢ) (e.g., first fluid and/or fluid from the first conduit) is greater than 0.1. In some embodiments, the ratio of the volumetric flow rate of the outer fluid (Qₒ) to the volumetric flow rate of the inner fluid (Qᵢ) is greater than or equal to 0.2, greater than or equal to 0.4, or greater than or equal to 0.6. In certain embodiments, the ratio of the volumetric flow rate of the outer fluid (Qₒ) to the volumetric flow rate of the inner fluid (Qᵢ) is less than or equal to 1.

In some embodiments, the outer fluid (e.g., second fluid and/or fluid from the second conduit) and inner fluid (e.g., first fluid and/or fluid from the first conduit) do not mix substantially in the needle and/or article, because mixing dilutes the inner fluid, reducing the benefits of axially lubricated flow. In certain embodiments, the timescale of convection is shorter than the time it takes for the inner fluid and outer fluid to mix substantially in the needle and/or article. In accordance with some embodiments, the outer fluid mixes with the inner fluid at most 50% while in the needle and/or article. That is, at most 50% of the outer fluid is mixed with the inner fluid while in the needle and/or article while the remainder of the outer fluid remains unmixed with the inner fluid. For example, in certain embodiments, the outer fluid mixes with the inner fluid at most 40%, at most 30%, at most 20%, or at most 10% while in the needle and/or article. According to certain embodiments, the percentage of mixing can be determined by visual inspection. In some embodiments, this could be accomplished by dyeing the inner fluid and/or outer fluid, taking photographs at the outlet of the needle and/or article, and measuring the extent of mixing of the two fluids from the diffusion and/or spreading of the dye(s). In certain embodiments, the extent of mixing could be measured at different lengths by cutting the needle to the length of interest, and photographing the fluids at the outlet.

In some embodiments, the inner fluid (e.g., first fluid and/or fluid from the first conduit) and the outer fluid (e.g., second fluid and/or fluid from the second conduit) comprise completely different components. For example, in some embodiments, the inner fluid and the outer fluid do not have any components in common. One such example would be if the inner fluid comprises a drug and water, while the outer fluid comprises an organic solvent.

In some embodiments, the inner fluid (e.g., first fluid and/or fluid from the first conduit) and the outer fluid (e.g., second fluid and/or fluid from the second conduit) comprise one or more components (e.g., a solvent and/or a buffer) that are the same. For example, in certain embodiments, the inner fluid and the outer fluid both comprise water.

In certain embodiments, the inner fluid (e.g., first fluid and/or fluid from the first conduit) and/or the outer fluid (e.g., second fluid and/or fluid from the second conduit) comprises one or more components that are different. For example, in some embodiments, the inner fluid comprises water and the outer fluid does not.

In certain embodiments, the inner fluid (e.g., first fluid and/or fluid from the first conduit) and the outer fluid (e.g., second fluid and/or fluid from the second conduit) comprise one or more components that are different and one or more components that are the same. For example, in some embodiments, the inner fluid and the outer fluid comprise the same components except that the inner fluid also has a drug (e.g., a biologic). For example, in certain embodiments, the inner fluid and the outer fluid both comprise water, but the inner fluid has a drug (e.g., a biologic) and the outer fluid does not. In some embodiments, the inner fluid and the outer fluid comprise exactly the same components (e.g., a buffer) except that one of the fluids (e.g., the inner fluid) has an additional component (e.g., a drug).

In some embodiments, the inner fluid (e.g., first fluid and/or fluid from the first conduit) and the outer fluid (e.g., second fluid and/or fluid from the second conduit) comprise exactly the same components (e.g., a buffer and a drug), but the concentrations of one or more of the components are different (e.g., a drug). For example, in some embodiments, the inner fluid and the outer fluid comprise exactly the same components (e.g., a buffer and a drug), but the concentration of one or more of the components (e.g., a drug) is higher in the inner fluid. As a skilled person would understand, in some embodiments, the different concentration of one or more of the components could result in different physical and/or chemical properties. For example, in an embodiment where the inner fluid has a high concentration of a biologic drug and the outer fluid has a low concentration of the biologic drug, but the inner and outer fluids are otherwise identical, the viscosity and/or density of the inner fluid may be much higher than that of the outer fluid.

In some embodiments, the molar concentration of one component (e.g., a drug) in the outer fluid (e.g., second fluid and/or fluid from the second conduit) is greater than or equal to 5%, greater than or equal to 10%, greater than or equal to 20%, greater than or equal to 30%, greater than or equal to 40%, greater than or equal to 50%, greater than or equal to 60%, greater than or equal to 75%, greater than or equal to 90%, or greater than or equal to 95% less than the molar concentration of that component in the inner fluid (e.g., first fluid and/or fluid from the first conduit). In some embodiments, the molar concentration of one component (e.g., a drug) in the outer fluid (e.g., second fluid and/or fluid from the second conduit) is less than or equal to 100%, less than or equal to 99%, less than or equal to 95%, less than or equal to 90%, less than or equal to 80%, less than or equal to 70%, less than or equal to 60%, or less than or equal to 50% less than the molar concentration of that component in the inner fluid (e.g., first fluid and/or fluid from the first conduit). Combinations of these ranges are also possible (e.g., greater than or equal to 5% and less than or equal to 100%, or greater than or equal to 10% and less than or equal to 50%). For example, if the molar concentration of the component was 1M in the inner fluid and 0.1M in the outer fluid, the molar concentration of the component in the outer fluid would be 90% less than that in the inner fluid.

When the inner fluid (e.g., first fluid and/or fluid from the first conduit) and the outer fluid (e.g., second fluid and/or fluid from the second conduit) are in concentric contact and are moving, one or more components of the inner fluid (e.g., a drug, such as a biologic) may begin to diffuse into the outer fluid. The radial position of the distinction between the inner and outer fluids (R(x)) is given by the following equation:

$$R ( x ) = {R}_{0} + \sqrt{\frac{Dx}{\overline{V}}} \qquad ( Equation 16 )$$

Where R₀ is the radius of the inner fluid at the beginning of any section of interest where the fluids are in contact, x is the axial position along the section, D is the diffusion coefficient of the component (e.g., a drug) in the outer fluid, and V̅ is the average velocity of the inner fluid. The extent of this diffusion can be validated by visualization as described elsewhere herein (e.g., by using dye molecules with the same diffusion coefficient as the component (e.g., drug)). As used herein, the radial position of the distinction between the inner and outer fluid (R(x)) means the distance between the center of the inner fluid and the distinction (e.g., boundary) between the inner and outer fluids. For example, when the inner fluid and the outer fluid first make contact and no diffusion has taken place, R(x) will be the same as R₀. However, as the fluids move through the system and the axial position (x) increases, R(x) will become larger than R₀, in some embodiments.

According to some embodiments, the outer fluid (e.g., second fluid and/or fluid from the second conduit) is a Newtonian fluid. For example, in accordance with certain embodiments, the viscous stresses arising from flow of the outer fluid at every point is linearly related to the local strain rate. Examples of suitable Newtonian fluids include water, a water-based solution, a buffer (e.g., a pharmaceutically acceptable buffer, such as a buffer used in a pharmaceutical product, such as a biologic), a formulation (e.g., a pharmaceutical formulation, such as a biologic formulation), saline, a biocompatible oil (e.g., squalene, a fluorinated oil (e.g., HFE-7500), mineral oil, and/or triglyceride oil), benzyl benzoate, a metabolizable oil, an immunologic adjuvant (e.g., MF59, AS02, AS03 and/or AS04), and/or safflower oil.

In accordance with certain embodiments, the outer fluid (e.g., second fluid and/or fluid from the second conduit) is a yield stress fluid. For example, according to some embodiments, the outer fluid deforms and/or flows only when subjected to a stress above a certain critical value specific to the yield stress fluid. Examples of suitable yield stress fluids include bone putty, hydrogels, hydrogel microbeads, and/or polymer solutions (example: polyethylene glycol).

In some embodiments, the needle and/or article comprise an interior surface.

In certain embodiments, at least a portion (e.g., at least 10%, at least 25%, at least 50%, at least 75%, at least 90%, or all) of the interior surface of the needle and/or article comprises a texture. For example, in some embodiments, the interior surface of the needle and/or article comprises a plurality of features. For example, in certain embodiments, the external surface of the conduit comprises milliscale, microscale, and/or nanoscale features. The texture may be used, in certain embodiments, to control the wettability of the surface. Any of a variety of features may be used. Non-limiting examples of protrusions include spherical or hemispherical protrusions. In some embodiments, the features comprise protrusions such as ridges, spikes, and/or posts. The features may be formed, for example, by etching away or otherwise removing material from which the surface is made, in some embodiments. In other embodiments, the features may be added to the surface (e.g., by depositing the features onto the interior surface of the needle and/or article, for example). The features may be made of material that is the same as or different from the material from which the interior surface is made. In certain embodiments, the features may be dispersed on the interior surface in a random (e.g., fractal) or patterned manner.

According to some embodiments, the maximum height of the milliscale features is greater than 100 micrometers and up to 1 millimeter, greater than 100 micrometers and up to 200 micrometers, from 200 micrometers to 300 micrometers, from 300 micrometers to 500 micrometers, from 500 micrometers to 700 micrometers, from 700 micrometers to 1 millimeter, from 1 millimeter to 3 millimeters, from 3 millimeters to 5 millimeters, and/or from 5 millimeters to 10 millimeters. Combinations of the above cited ranges are also possible (e.g., from 300 micrometers to 700 micrometers, or from 200 micrometers to 1 millimeter).

According to some embodiments, the maximum height of the microscale features is from 1 micrometer to 10 micrometers, 10 micrometers to 20 micrometers, 20 micrometers to 30 micrometers, 30 micrometers to 50 micrometers, 50 micrometers to 70 micrometers, or 70 micrometers to 100 micrometers. Combinations of the above cited ranges are also possible (e.g., 30 micrometers to 70 micrometers, or 20 micrometers to 100 micrometers).

According to some embodiments, the maximum height of the nanoscale features is from 1 nm to 100 nm, 100 nm to 200 nm, 200 nm to 300 nm, 300 nm to 500 nm, 500 nm to 700 nm, or 700 nm to 1 micrometer. Combinations of the above cited ranges are also possible (e.g., 300 nm to 700 nm, or 200 nm to 1 micrometer).

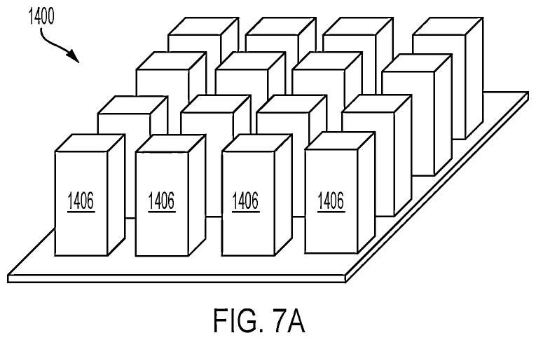

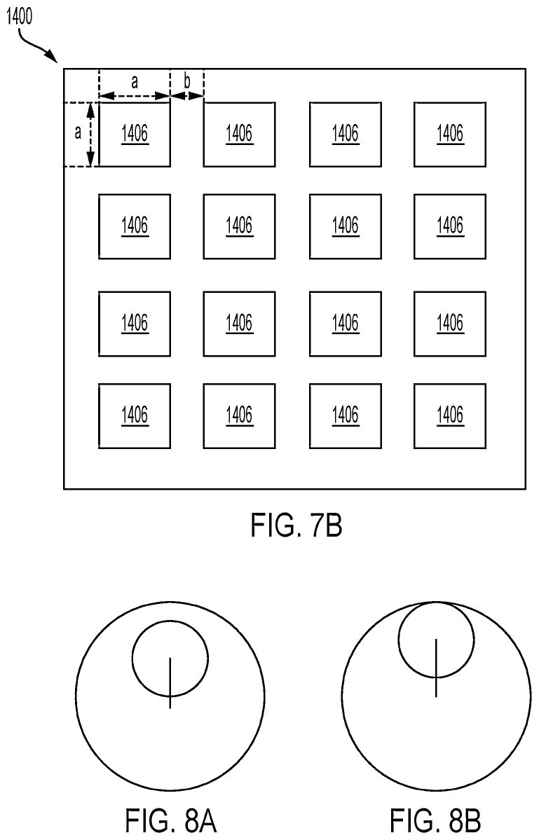

According to certain embodiments, the features (e.g., the milliscale, microscale, and/or nanoscale features) are distributed over the interior surface of the needle and/or the article such that the features occupy a particular solid fraction of the interior surface. The term “solid fraction” (also referred to as φₛ) occupied by a plurality of features on a surface, as used herein, refers to the area fraction of the surface that is occupied by the features. The solid fraction can be calculated by dividing the sum of the areas that the features occupy on the interior surface by the geometric surface area of the interior surface over which those features are distributed. For example, referring to FIGS. 7A-7B, interior surface portion 1400 (e.g., a portion of the interior surface of the needle and/or article) comprises a plurality of features 1406. Features 1406 in FIGS. 7A-7B are squares with side lengths a, and thus, each occupies an area on the interior surface equal to a². The remaining area of the interior surface is not occupied by features. In the set of embodiments illustrated in FIGS. 7A-7B, each of features 1406 have identical side lengths a and identical nearest neighbor spacings b. Accordingly, the surface solid fraction (φₛ) occupied by the features in FIGS. 7A-7B would be calculated as follows:

$$φ s = {a}^{2} / {( a + b )}^{2} \qquad ( Equation 17 )$$

In certain embodiments, the interior surface of the needle and/or article comprises a texture for which the solid fraction (φs) is less than or equal to 0.5. In some embodiments, the interior surface of the needle and/or article comprises a texture for which the solid fraction (φs) is less than or equal to 0.25 or less than or equal to 0.1.

In certain embodiments, a third fluid (in addition to the inner fluid and the outer fluid) can be impregnated between the features on the interior surface of the needle and/or article. The third fluid may, in some embodiments, be stably contained between the features such that the third fluid remains contained between the features while the inner and outer fluids are transported through the needle (and/or the article). The third fluid can be stably contained between the features, for example, by spacing the features sufficiently close such that the third liquid is stably contained between the features (e.g., via surface tension forces). In certain embodiments, the third fluid is contained between the features but does not cover the tops of the features. In some embodiments, the properties of the third fluid may be tailored to control the wettability of the interior surface of the needle and/or article.

In accordance with some embodiments, for a given inner fluid, outer fluid, and interior textured surface of the needle and/or article, the spreading coefficient (Sₒₙ₍ᵢ₎) is greater than or equal to 0. In some embodiments, the texture imparts wettability for at least one fluid (e.g., the outer fluid) when a droplet of that fluid is present on the interior surface of the needle and/or article in another fluid (e.g., the inner fluid). That is, in certain instances, the at least one fluid (e.g., the outer fluid) is wetting when the texture is present, but would not be wetting in an identical system without the texture.

According to certain embodiments, at least a portion (e.g., at least 10%, at least 25%, at least 50%, at least 75%, at least 90%, or all) of the interior surface of the needle and/or article comprises a coating. For example, in some embodiments, the interior surface of the needle and/or article comprises a conformal, smooth coating with limited discontinuities. In some embodiments, a conformal, smooth coating with limited discontinuities has less than or equal to 10⁸, less than or equal to 10⁶, or less than or equal to 10⁴ discontinuities/m². A coating is considered to be conformal if 90% of the facial area of the coating is within 20% of the average thickness of the coating. In accordance with some embodiments, for the inner fluid, the outer fluid, and the interior surface of the coating, the spreading coefficient (Sₒₙ₍ᵢ₎) is greater than or equal to 0. In some embodiments, the coating imparts wettability for at least one fluid (e.g., the outer fluid) when a droplet of that fluid is present on the interior surface of the needle and/or article in the other fluid (e.g., inner fluid). That is, in certain instances, the at least one fluid (e.g., the outer fluid) is wetting when the coating is present, but would not be wetting in an identical system without the coating.

The needle can have, in accordance with certain embodiments, any of a variety of lengths. Certain of the embodiments described herein can be used to achieve stable core sheath flow within a needle having a relatively long length. According to certain embodiments, the needle has a length of greater than or equal to 5 microns, greater than or equal to 10 microns, greater than or equal to 25 microns, greater than or equal to 50 microns, greater than or equal to 100 microns, greater than or equal to 1 mm, greater than or equal to 5 mm, greater than or equal to 10 mm, or greater than or equal to 100 mm. According to some embodiments, the needle has a length of less than or equal to 250 mm, less than or equal to 100 mm, less than or equal to 50 mm, less than or equal to 10 mm, less than or equal to 5 mm, less than or equal to 1 mm, less than or equal to 500 microns, less than or equal to 100 microns, less than or equal to 50 microns, or less than or equal to 25 microns. Combinations of these ranges are also possible (e.g., 5 microns to 5 mm or 5 mm to 10 mm).

It should be understood that the use of relatively long needles is not required, and that in other embodiments, the needle is relatively short. For example, in some embodiments, the needle has a length of less than 5 mm, less than or equal to 1 mm, less than or equal to 500 microns, or less than or equal to 100 microns.

In certain embodiments, the needle is narrow. For example, in some cases, the needle has an inner diameter of greater than or equal to 5 microns, greater than or equal to 10 microns, greater than or equal to 25 microns, greater than or equal to 50 microns, greater than or equal to 100 microns, greater than or equal to 250 microns, greater than or equal to 500 microns, or greater than or equal to 750 microns. In some embodiments, the needle has an inner diameter of less than or equal to 1 mm, less than or equal to 750 microns, less than or equal to 500 microns, less than or equal to 310 microns, less than or equal to 250 microns, less than or equal to 100 microns, less than or equal to 50 microns, less than or equal to 25 microns, or less than or equal to 10 microns. Combinations of these ranges are also possible (e.g., greater than or equal 5 microns and less than or equal to 1 mm, or greater than or equal to 10 microns and less than or equal to 310 microns).

Methods are also described herein. In some embodiments, the method comprises initiating flow of at least a portion (e.g., at least 50%, at least 75%, at least 90%, or all) of an inner fluid (e.g., an inner fluid described herein) (e.g., first fluid and/or fluid from the first conduit) within an article described herein. According to some embodiments, at least a portion (e.g., at least 50%, at least 75%, at least 90%, or all) of the inner fluid is transported from the article to the needle. In certain embodiments, at least a portion (e.g., at least 50%, at least 75%, at least 90%, or all) of the inner fluid is ejected from the needle.

In certain embodiments, the method comprises initiating flow of a least a portion (e.g., at least 50%, at least 75%, at least 90%, or all) of an outer fluid (e.g., an outer fluid described herein) (e.g., second fluid and/or fluid from the second conduit) within an article described herein. According to some embodiments, at least a portion (e.g., at least 50%, at least 75%, at least 90%, or all) of the outer fluid is transported from the article to the needle. In certain embodiments, at least a portion (e.g., at least 50%, at least 75%, at least 90%, or all) of the outer fluid is ejected from the needle.

In some embodiments, it is beneficial for lower amounts of the outer fluid (e.g., second fluid and/or fluid from the second conduit) to be ejected compared to the amount of inner fluid (e.g., first fluid and/or fluid from the first conduit) ejected (e.g., such that a patient is not exposed to large amounts of a lubricating fluid). According to certain embodiments, the ratio of a volume of the inner fluid ejected from the needle and/or article to the total volume (e.g., inner fluid and outer fluid) ejected from the needle and/or article (Φ) (the volume fraction) is greater than or equal to 0.5. greater than or equal to 0.6. greater than or equal to 0.7, greater than or equal to 0.8, or greater than or equal to 0.9. The volume fraction (Φ) can also be expressed as:

$$Φ = {Q}_{i} / ( {Q}_{i} + {Q}_{o} ) \qquad ( Equation 18 )$$

In accordance with some embodiments, when the inner fluid (e.g., first fluid and/or fluid from the first conduit) has a certain capillary number and the outer fluid (e.g., second fluid and/or fluid from the second conduit) has a certain capillary number, axially lubricated flow may be observed, whereas viscous displacement may otherwise be observed.

According to certain embodiments, the capillary number of the inner fluid (e.g., first fluid and/or fluid from the first conduit) is greater than or equal to 0.01, greater than or equal to 0.1, greater than or equal to 1, greater than or equal to 10, greater than or equal to 20, or greater than or equal to 25. In some embodiments, the capillary number of the inner fluid is less than or equal to 30, less than or equal to 25, less than or equal to 10, less than or equal to 1, or less than or equal to 0.1. Combinations of these ranges are also possible (e.g., greater than or equal to 0.01 and less than or equal to 30).

In some embodiments, the capillary number of the outer fluid (e.g., second fluid and/or fluid from the second conduit) is greater than or equal to 0.001, greater than or equal to 0.01, greater than or equal to 0.1, greater than or equal to 1, greater than or equal to 10, or greater than or equal to 20. In certain embodiments, the capillary number of the outer fluid is less than or equal to 25, less than or equal to 10, less than or equal to 1, less than or equal to 0.1, or less than or equal to 0.01. Combinations of these ranges are also possible (e.g., 0.001-25).

In certain embodiments, the capillary number of the inner fluid is larger than the capillary number of the outer fluid. The capillary number of a fluid is expressed as:

$${C}_{a} = \frac{( μ * V )}{σ} \qquad ( Equation 19 )$$

where μ (mu) is the dynamic viscosity of the fluid, V is the average linear velocity of the fluid, and σ (sigma) is the interfacial tension between the inner and outer fluids.

In some embodiments, the orientation of the system (e.g., the needle and/or article) affects the timescale of eccentricity. For example, in accordance with certain embodiments, a T_{c}/tₑ of less than or equal to 1 is easier to achieve with the system (e.g., the needle and/or article) closer to vertical (90° from a line perpendicular to gravity), and more difficult to achieve closer to horizontal (0° from a line perpendicular to gravity).

In accordance with some embodiments, the longitudinal axis of the needle is within 45 degrees of a line perpendicular to gravity for at least one period of time. For example, in some cases, the longitudinal axis of the needle is within 30 degrees, 15 degrees, or 0 degrees of a line perpendicular to gravity for at least one period of time. In some embodiments, the period of time is between initiating flow of the inner fluid and/or outer fluid and ejection of the inner fluid and/or outer fluid from the needle. For example, in certain cases, the period of time is at least a portion of time (e.g., at least 50%, at least 75%, at least 90%, or the entirety of the time) between the initiating flow and the ejection from the needle.

As discussed above, in some embodiments, it is beneficial for lower amounts of the outer fluid (e.g., second fluid and/or fluid from the second conduit) to be ejected compared to the amount of inner fluid (e.g., first fluid and/or fluid from the first conduit) ejected (e.g., such that a patient is not exposed to large amounts of a lubricating fluid). In certain embodiments, the volumetric flow rate of the inner fluid is greater than the volumetric flow rate of the outer fluid. According to some embodiments, the volumetric flow rate of the inner fluid is ≥10⁻²×γπdₙ²/μᵢ. For example, in certain cases, the volumetric flow rate of the inner fluid is ≥5×10⁻²×γπdₙ²/μᵢ or ≥10⁻¹×γπdₙ²/μᵢ. In accordance with certain embodiments, the volumetric flow rate of the outer fluid is ≥10⁻³×γπdₙ²/μₒ. For example, in some instances, the volumetric flow rate of the outer fluid is ≥10⁻³×γπdₙ²/μₒ. For the volumetric flow rate, dₙ is the diameter of the needle, γ (gamma) is the surface tension of the two fluids, and μ is the dynamic viscosity of the fluid (where the i denotes the inner fluid and the o denotes the outer fluid).

In some embodiments, the concentration of a solubilized or suspended species (e.g., a drug) in the inner fluid (e.g., first fluid and/or fluid from the first conduit) can be significantly larger than in an identical article, system, and/or method without the outer fluid axially surrounding the inner fluid (and/or an identical system and/or method without the article). For example, in some cases, the ratio of the concentration of a solubilized or suspended species (e.g., a drug) in the inner fluid according to certain embodiments disclosed herein compared to an identical article, system, and/or method without the outer fluid axially surrounding the inner fluid (and/or an identical system and/or method without the article) is greater than or equal to 1.1:1, greater than or equal to 1.5:1, greater than or equal to 2:1, greater than or equal to 5:1, greater than or equal to 10:1, greater than or equal to 50:1, greater than or equal to 100:1, or greater than or equal to 250:1. In some embodiments, the ratio of the concentration of a solubilized or suspended species (e.g., a drug) in the inner fluid according to certain embodiments disclosed herein compared to an identical article, system, and/or method without the outer fluid axially surrounding the inner fluid (and/or an identical system and/or method without the article) is less than or equal to 500:1, less than or equal to 250:1, less than or equal to 100:1, less than or equal to 50:1, less than or equal to 10:1, less than or equal to 5:1, or less than or equal to 2:1. Combinations of these ranges are also possible (e.g., 1.1:1 to 500:1).

In some embodiments, the articles, systems, and/or methods disclosed herein have a reduced pressure during injection compared to an identical article, system, and/or method without the outer fluid axially surrounding the inner fluid (and/or an identical system and/or method without the article). For example, in some cases, the ratio of the pressure during injection compared to that of an identical article, system, and/or method without the outer fluid axially surrounding the inner fluid (and/or an identical system and/or method without the article) is less than or equal to 0.9:1, less than or equal to 0.7:1, less than or equal to 0.5:1, less than or equal to 0.3:1, less than or equal to 0.1:1, or less than or equal to 0.01:1. In some embodiments, the ratio of the pressure during injection compared to an identical article, system, and/or method without the outer fluid axially surrounding the inner fluid (and/or an identical system and/or method without the article) is greater than or equal to 0.001:1, greater than or equal to 0.01:1, or greater than or equal to 0.1:1. Combinations of these ranges are also possible (e.g., 0.001:1 to 0.9:1 or 0.1:1 to 0.3:1).

Certain of the embodiments disclosed herein can provide one or more of several benefits, including reduced contamination, reduced needle clogging, reduced protein inactivation (e.g., when the inner fluid comprises a protein), increased concentrations of formulations (e.g., the inner fluid may be a high concentration drug formulation), increased viscosity of fluids, increased feasibility of subcutaneous administration (rather than intravenous administration), smaller needles, shorter injection times, reduced pain, fewer doses, reduced hydrodynamic resistance in the needle, reduced shear forces on the inner fluid, and/or reduced pressures. Examples of benefits that may arise from subcutaneous administration (which frequently require higher concentrations) rather than intravenous administration, in some embodiments, include increased feasibility of self-administration, reduced hospitalization, reduced treatment costs, and/or increased patient compliance.

In some embodiments, the systems and/or articles described herein can inject viscous fluids without the use of larger needle gauges or prolonged injection times, which can cause pain. Moreover, in certain embodiments, the systems and/or articles described herein can inject high concentration formulations without the use of syringe pumps, which can cause pain and can require a hospital setting. Additionally, in accordance with some embodiments, the systems and/or articles described herein can inject viscous fluids without the use of needle free jet injectors, which frequently result in contamination and high costs. Further, in accordance with certain embodiments, the systems and/or articles described herein can inject viscous fluids without particle encapsulation, which frequently results in protein inactivation, density based separation, needle clogging, and a higher degree of manufacturing complexity. The lack of a practical methodology to inject high viscosity formulations has not only limited the applicability of subcutaneous biologic formulations, but also hinders the development of new formulations as developers are forced to design formulations with lower viscosities. Therefore, there remains a pressing need to achieve injectability through a simple and inexpensive injection technique with minimal additions to the pharmaceutical manufacturing process and without risk of cross contamination.

Various of the components described herein are “fluidically connected” to or “in fluidic communication” with other components. Generally, two components are fluidically connected and/or in fluidic communication when a connection exists between them such that fluid could flow and/or be transported from one to the other. In some cases, any two components that are described as “fluidically connected” or “in fluidic communication” may be directly fluidically connected or in direct fluidic communication, meaning that there are no components (e.g., a conduit or segment) between them. In certain instances, any two components that are described as “fluidically connected” or “in fluidic communication” may be indirectly fluidically connected or in indirect fluidic communication, meaning that there is one or more components (e.g., a conduit or segment) between them that does not prevent fluid from flowing and/or being transported from one to the other.

The following examples are intended to illustrate certain embodiments of the present invention, but do not exemplify the full scope of the invention.

### EXAMPLE 1

This example demonstrates the reduction of eccentricity in the article (and/or needle) by changing the dimensions and/or geometry of the article.

FIG. 3A shows a cross-sectional schematic that is an example of one possible design for an article fluidically connected to a needle, in accordance with certain embodiments, with different dimensions labeled: D_{Hi}—Inner fluid outlet's inner diameter in the hub; D_{Ho}—Outer fluid outlet's inner diameter in the hub; D_{C}—Inner diameter of the hub connector; D_{N}—Inner diameter of the needle; L_{HFD}—fully developed flow length in the hub; L_{HPC}—Pre-constriction flow length in the hub; L_{HC}—Constriction length in the hub; L_{CFD}—fully developed flow length in the connector; L_{CPC}—Pre-constriction flow length in the connector; and L_{NFD}—fully developed flow length in the needle.

It was determined that throughout this system, convection and buoyancy driven eccentricity were the main competing timescales. Convection transports the fluids through any given section of the system while buoyancy forces a growth in the eccentricity parameter E. FIGS. 3B and 3C show two examples of designs where the D_{Ho} was taken from 4 mm (FIG. 3B) to 2 mm (FIG. 3C). In these examples, glycerol (40 cP) was used as a model inner fluid and a solution of salt water (1.6 cP) was used as the outer fluid. In FIG. 3B, the flow became fully eccentric (E=1) as the large D_{HO} resulted in a low flow velocity and hence a large T_{c}. This state of full eccentricity was irreversible and was maintained in the needle as shown in the bottom image of FIG. 3B. In contrast, in FIG. 3C, a reduction in the D_{HO} resulted in a reduced T_{c}. This allowed touchdown of the inner fluid (E=1) to be avoided and the flow was maintained at E=0 throughout the article and in the needle. In the needle, T_{c} was already less than the tₑ for these liquids without any modifications and thus, preventing touchdown and eccentricity in the article ensured concentric coaxial lubrication in the needle.

Preventing inner fluid touchdown (E=1) also provided robust eccentricity reduction in the needle. In FIG. 4A, the flow was partially eccentric in the article but the addition of the constriction region of the article reduced the eccentricity and brought it back to E=0 in the needle, similar to in the case of FIG. 4B where flow was concentric throughout the article and the needle. Since pressure reduction performance is greatly tied to the extent of eccentricity, utilizing constrictions to minimize eccentricity can greatly enhance performance.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

Unless clearly indicated to the contrary, the flowrates (Q) described herein are volumetric flow rates.

Unless clearly indicated to the contrary, the viscosities (μ) described herein are dynamic viscosities. The dynamic viscosity of a fluid can be determined using a TI ARG-2 rheometer, varying the shear rate from 10s−1 to 500s−1.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a fluidic pathway comprising an inlet and an outlet and configured to receive a first fluid and a second fluid;
wherein a cross-sectional area of the inlet is larger than a cross-sectional area of the outlet; and
wherein the article is configured such that the second fluid axially surrounds the first fluid in the article with an eccentricity parameter of less than 1.

2. The article of claim 1, wherein the article is configured such that the eccentricity parameter of the first and second fluids is maintained or lower directly downstream of the outlet than the highest eccentricity parameter at any segment of the article.

3. An article, comprising:
a fluidic pathway comprising an inlet and an outlet and configured to receive a first fluid and a second fluid;
wherein a cross-sectional area of the inlet is larger than a cross-sectional area of the outlet;
wherein the article is configured such that the second fluid axially surrounds the first fluid in the article; and
wherein the article is configured such that the eccentricity parameter of the first and second fluids is maintained or lower directly downstream of the outlet than the highest eccentricity parameter at any segment of the article.

4. The article of claim 3, wherein the article is configured such that the second fluid axially surrounds the first fluid in the article with an eccentricity parameter of less than 1.

5. The article of claim 1, wherein the eccentricity parameter in the article is less than or equal to 0.9.

6. The article of claim 1, wherein the timescale of convection is less than the timescale of eccentricity in the article, where the timescale of convection is how long it takes for the first and second fluids to travel through the article and the timescale of eccentricity is the time for spatially stable eccentricity to arise in the article.

7. The article of claim 1, wherein the article is configured such that the eccentricity parameter of the first and second fluids is greater than or equal to 10% lower directly downstream of the outlet than the highest eccentricity parameter at any segment of the article.

8. The article of claim 1, where the difference in the density of the first fluid and the density of the second fluid is less than or equal to 400 kg/m³.

9. The article of claim 1, wherein the article comprises one or more constricted regions, protrusions on an inner surface, ribs on an inner surface, and/or fins on an inner surface.

10. The article of claim 1, wherein the article comprises a tapered region.

11. The article of claim 1, wherein the article has a ratio of L_{HPC}/D_{HO} of less than or equal to 2.

12. The article of claim 1, wherein the article comprises a connector region and has a ratio of L_{CPC}/D_{c} of less than or equal to 2.

13. The article of claim 1, wherein the article contains the first fluid and the second fluid, and wherein the length (L) and diameter (D) of at least a portion of the article satisfies the following equation for the first fluid and the second fluid:
$$\frac{L π {D}^{2}}{4 {Q}_{avg} \sqrt{\frac{D [ 1 - \frac{1}{\sqrt{2 - \frac{{μ}_{0}}{{μ}_{i}}}} ]}{\vert g cos ( θ ) ( 1 - \frac{{ρ}_{o}}{{ρ}_{i}} ) \vert}}} < 1$$
where ρₒ is the density of the second fluid, ρᵢ is the density of the first fluid, μₒ is the viscosity of the second fluid, μᵢ is the viscosity of the first fluid, Q_{avg} is the average flowrate of the first fluid and the second fluid, L is the length of the portion of the article, θ is the angle between the length of the portion of the article and the horizontal plane, g is the gravitational constant, and D is the average diameter of the portion of the article.

14. The article of claim 1, wherein the article contains the first fluid and the second fluid, and wherein the length (L) and diameter (D) of at least a portion of the article satisfies the following equation for the first fluid and the second fluid:
$$\frac{LAi}{Qi \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$
where ρₒ is the density of the second fluid, ρᵢ is the density of the first fluid, μₒ is the viscosity of the second fluid, μᵢ is the viscosity of the first fluid, Qi is the flowrate of the first fluid through the portion of the article, L is the length of the portion of the article, θ is the angle between the length of the portion of the article and the horizontal plane, g is the gravitational constant, D is the average diameter of the portion of the article, and Aᵢ is determined by the following equations:
$${r}_{i}^{*} = \frac{{r}_{o}}{\sqrt{2 - {μ}_{o} / {μ}_{i}}} \\ {A}_{i} = {π ( {r}_{i}^{*} )}^{2} ,$$
where rᵢ* is the optimal radius of the first fluid, μₒ is the dynamic viscosity of the second fluid, μᵢ is the dynamic viscosity of the first fluid, rₒ is the radius of the second fluid, and Aᵢ is the cross-sectional area of the first fluid as it flows through the portion of the article.

15. The article of claim 1, wherein the article contains the first fluid and the second fluid, and wherein the length (L) and diameter (D) of at least a portion of the article satisfies the following equation for the first fluid and the second fluid:
$$\frac{L π {D}^{2}}{4 {Q}_{total} \sqrt{\frac{D [ 1 - \frac{1}{\sqrt{2 - \frac{{μ}_{o}}{{μ}_{i}}}} ]}{\vert g cos ( θ ) ( 1 - \frac{{ρ}_{o}}{{ρ}_{i}} ) \vert}}} \leq 1$$
where ρₒ is the density of the second fluid, ρᵢ is the density of the first fluid, μₒ is the viscosity of the second fluid, μᵢ is the viscosity of the first fluid, Qₜₒₜₐₗ is the total flowrate of both fluids through the portion of the article, L is the length of the portion of the article, θ is the angle between the length of the portion of the article and the horizontal plane, g is the gravitational constant, and D is the average diameter of the portion of the article.

16. A system comprising the article of claim 1 and a needle fluidically connected to the outlet of the article.

17. A system comprising the article of claim 1 and a first conduit and a second conduit, wherein the first conduit and second conduit are fluidically connected to the inlet of the article.

18. The system of claim 17, wherein the system further comprises a needle fluidically connected to the outlet of the article.

19. The system of claim 17, wherein the first conduit is arranged in a side-by-side configuration with the second conduit.

20. The system of claim 19, wherein the system further comprises a chamber comprising a first internal volume and a second internal volume, wherein the first internal volume is fluidically connected to the first conduit and the inlet of the article, and the second internal volume is fluidically connected to the second conduit and the inlet of the article.

21. The system of claim 17, wherein the second conduit axially surrounds the first conduit.

22. The system of claim 17, wherein the system further comprises:
a first plunger associated with the first conduit; and
a second plunger associated with the second conduit.

23. The system of claim 22, wherein the system further comprises a solid body connecting the first plunger and the second plunger.

24. The system of claim 22, wherein the system is configured such that when the first plunger and the second plunger are compressed, fluid within the first conduit is transported to the article and fluid within the second conduit is transported to the article such that the fluid from the second conduit at least partially axially surrounds fluid from the first conduit in the article.

25. The system of claim 22, wherein the system is configured such that when the first plunger and the second plunger are compressed, fluid within the first conduit is transported to the needle and fluid within the second conduit is transported to the needle such that the fluid from the second conduit at least partially axially surrounds fluid from the first conduit in the needle.

26. A method of delivering one or more fluids using the article of claim 1.
